(19) United States
(12) Patent Application Publication
(10) Pub. No.: US 2010/0105673 A1
(43) Pub. Date: Apr. 29, 2010

(54) SUBSTITUTED AMINOFURANONES AND THEIR USE

(75) Inventors: Adrian Tersteegen, Wuppertal (DE); Dirk Heimbach, Dusseldorf (DE); Kai Thede, Wuppertal (DE); Reinhold Welker, Bensheim (DE); Beate Fast, Ludwigshafen (DE); Arnold Paessens, Haan (DE); Frank Dittmer, Dusseldorf (DE); Rudolf Schohe-Loop, Wuppertal (DE); Axel Harrenga, Wuppertal (DE); Alexander Hillisch, Velbert (DE); Kerstin Henninger, Wuppertal (DE); Walter Hübsch, Wuppertal (DE); Marcus Bauser, Wuppertal (DE); Susanne Greschat, Dusseldorf (DE); Dirk Schneider, Wuppertal (DE); Tobias Marquardt, Berlin (DE); Andreas Göller, Wuppertal (DE); Andreas Urban, Hattingen (DE); Steffen Wildum, Schwelm (DE); Daniela Paulsen, Wuppertal (DE)

Correspondence Address: EDWARDS ANGELL PALMER & DODGE LLP, P.O. BOX 55874, BOSTON, MA, 02205, US

(73) Assignee: Bayer Schering Pharma Aktiengesellschaft, Berlin (DE)

(21) Appl. No.: 12/518,717

(22) PCT Filed: Dec. 8, 2007
(86) PCT No.: PCT/EP07/10712

§ 371 Date: Dec. 8, 2009

(30) Foreign Application Priority Data
Dec. 15, 2006 (DE) 10 2006 059 319.7

(51) Int. Cl. A61K 31/365; A61K 31/5377; A61K 31/438; C07D 413/12; C07D 491/107; C07D 307/94; A61P 31/18; A61P 31/12

(52) U.S. Cl. 514/231.5; 514/278; 514/462; 544/230; 546/16; 549/265

(57) ABSTRACT

The present invention relates to novel substituted aminofurans, to methods for their preparation, to their use in the treatment and/or prophylaxis of diseases as well as their use in the manufacture of medical products for the treatment and/or prophylaxis of diseases, in particular retroviral disorders, in humans and/or animals.

The present invention relates to novel substituted aminofuranones, methods for their preparation, their use for the treatment and/or prophylaxis of diseases as well as their use for the manufacture of medicaments for the treatment and/or prophylaxis of diseases, in particular retroviral diseases, in humans and/or animals.

HIV (virus of human immune deficiency) causes a chronically persistent and progressive infection. The disease runs through different stages from asymptomatic infection up to the clinical picture AIDS (Acquired Immunodeficiency Syndrome). AIDS is the final stage of the disease caused by infection. Characteristic of HIV/AIDS disease is the long clinical latency period with persistent viremia which in the final stage leads to the failure of the immune defense.

Through the introduction of anti-HIV combination therapy in the 1990s it was possible to slow sustainably the progression of the disease and thus to increase substantially the life expectancy of HIV-infected patients (Palella et al., N. Engl. J. Med. 1998, 238, 853-860).

The anti-HIV substances currently on the market inhibit the replication of the HI virus by inhibition of the essential viral enzymes reverse transcriptase (RT), the protease or the HIV fusion (review in Richman, Nature 2001, 410, 995-1001). There are two classes of RT inhibitors: nucleosidic RT inhibitors (NRTI) act through competitive inhibition or chain termination during DNA polymerization. Non-nucleosidic RT inhibitors (NNRTI) bind allosterically to a hydrophobic pocket in the vicinity of the active centre of the RT and induce conformational changes in the enzyme. The currently available protease inhibitors (PI) on the other hand block the active centre of the viral protease and thus prevent the maturation of newly formed particles to infectious virions.

Since monotherapy with the currently available anti-HIV medicaments leads within a very short time to therapy failure through the selection of resistant viruses, normally a combination therapy with several anti-HIV substances from different classes is undertaken (highly active antiretroviral therapy=HAART; Carpenter et al., J. Am. Med. Assoc. 2000, 283, 381-390).

In spite of the advances in anti-retroviral chemotherapy more recent studies show that an eradication of HIV and an associated cure of the HIV infection is not to be expected with the available medicaments: latent virus remains in dormant lymphocytes and represents a reservoir for reactivation and thus for a renewed virus proliferation (Finzi et al., Nature Med. 1999, 5, 512-517; Ramratnam et al., Nature Med. 2000, 6, 82-85). HIV-infected patients are thus dependent on an efficient antiviral therapy throughout their lifetime. In spite of combination therapy a selection of resistant viruses occurs after a certain time. Since characteristic resistance mutations accumulate for every therapeutic class the failure of one therapy often means a loss of efficacy of the complete substance class.

The occurrence of resistance is usually favoured by the poor compliance of the patient, which is brought about by an unfavourable side effect profile and complicated dosing regime of the anti-HIV medicaments.

Thus there is urgent need for new therapeutic options for combating HIV infections. For this the identification of new chemical lead structures is important and a pressing objective of HIV therapy research, which addresses either a new target in the replication of HIV and/or are active against the growing number of resistant clinical HIV isolates.

JP 61100577 and T. Hiyama, et al., Bull. Chem. Soc. Jpn. (1987), 60(6) 2139-50 describe aminofuranones as synthetic building blocks for the preparation of antihypertensives and the anti-tumour antibiotic basilidin. The synthesis of aminofuranones is described by T. Hiyama, et al., Tetrahedron Lett. (1985), 26(20), 2459-62.

The invention relates to compounds of formula

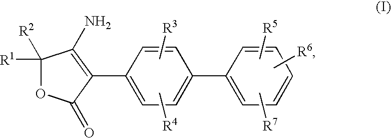

in which
R¹ and R² together with the carbon atom to which they are bonded form a group of formula

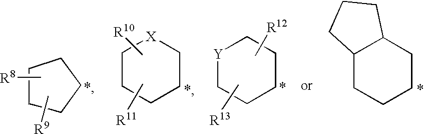

whereby
* represents the carbon atom to which R¹ and R² are bonded,
n represents the number 1, 2 or 3,
X represents an oxygen atom, a sulphur atom or NR¹⁴,
   whereby
   R¹⁴ represents C₁-C₆ alkyl, C₂-C₄ alkenyl, C₁-C₄ alkylsulphonyl, benzylsulphonyl, —(CH₂)ₒCOR¹⁶, —(CH₂)ₚCONR¹⁷R¹⁸, —(CH₂)_{q}NR²⁴COR²⁵ or —(CH₂)ᵥNR²⁶SO₂R²⁷,
      whereby alkyl, alkenyl and alkylsulphonyl can be substituted with 1 to 2 substituents, whereby the substituents are selected independently of one another from the group consisting of halogen, cyano, hydroxy, trifluoromethyl, hydroxycarbonyl, aminosulphonyl, C₁-C₄ alkoxy, C₁-C₄ alkoxycarbonyl, C₁-C₆ alkylaminocarbonyl, C₁-C₆ alkylaminosulphonyl, benzylaminosulphonyl, C₃-C₇ cycloalkyl, phenyl, 5- to 10-membered heterocyclyl, 5- to 10-membered heteroaryl and —OR²²,
         wherein phenyl, heterocyclyl and heteroaryl can be substituted with 1 to 3 substituents, whereby the substituents are selected independently of one another from the group consisting of halogen, cyano, nitro, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl, C₁-C₄ alkoxycarbonyl and benzyl,
         and
         wherein alkoxy can be substituted with a substituent selected from the group consisting of halogen, cyano, trifluoromethyl, hydroxy, hydroxycarbonyl, aminocarbonyl, aminosulphonyl, C₁-C₄ alkoxy, C₁-C₄ alkoxycarbonyl, C₁-C₄ alkylaminocarbonyl, C₁-C₄ alkylaminosulphonyl, C₃-C₇ cycloalkyl, phenyl, 5- to 10-membered heterocyclyl and 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
         and
         R²² represents C₃-C₇ cycloalkyl, phenyl, 5- to 10-membered heterocyclyl or 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      and
      whereby
      represents a number 0, 1, 2 or 3,
      p represents a number 0, 1, 2 or 3,
      q represents a number 2 or 3,
      v represents a number 2 or 3,
      R¹⁶ represents C₁-C₆ alkyl, C₂-C₄ alkenyl, C₁-C₆ alkoxy, phenyl, benzyloxy or 5- to 10-membered heterocyclyl,
         whereby alkyl, alkenyl and alkoxy can be substituted with a substituent, whereby the substituent is selected from the group consisting of halogen, cyano, hydroxy, trifluoromethyl, hydroxycarbonyl, aminosulphonyl, C₁-C₄ alkoxy, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      R¹⁷ represents hydrogen, C₁-C₄ alkyl or phenyl,
         whereby alkyl can be substituted with a substituent, whereby the substituent is selected from the group consisting of methoxy, methoxycarbonyl, C₃-C₇ cycloalkyl, phenyl, 5- to 10-membered heterocyclyl and 5- to 10-membered heteroaryl,
         wherein cycloalkyl, phenyl, heterocyclyl and heteroaryl for their part can be substituted with 1 to 3 substituents, whereby the substituents are selected independently of one another from the group consisting of halogen, cyano, hydroxy, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl, C₁-C₄ alkylsulphonylamino and C₁-C₄ alkoxycarbonyl,
      R¹⁸ represents hydrogen or C₁-C₄ alkyl,
      R²⁴ represents hydrogen or C₁-C₄ alkyl,
      R²⁵ represents C₁-C₆ alkyl, C₂-C₄ alkenyl, C₁-C₆ alkoxy, phenyl, benzyloxy or 5- to 10-membered heterocyclyl,
         whereby alkyl and alkenyl an be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      R²⁶ represents hydrogen or C₁-C₄ alkyl,
      R²⁷ represents C₁-C₆ alkyl, C₂-C₄ alkenyl, phenyl or 5- to 10-membered heterocyclyl,
         whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
Y represents an oxygen atom, a sulphur atom or NR¹⁵,
   whereby
   R¹⁵ represents C₁-C₆ alkyl, C₂-C₄ alkenyl, C₁-C₄ alkylsulphonyl, benzylsulphonyl, —(CH₂)ᵣCOR¹⁹, —(CH₂)ₛCONR²⁰R²¹, —(CH₂)ₜNR²⁸COR²⁹ or —(CH₂)_{w}NR³⁰SO₂R³¹,
      whereby alkyl, alkenyl and alkylsulphonyl can be substituted with 1 to 2 substituents, whereby the substituents are selected independently of one another from the group consisting of halogen, cyano, hydroxy, trifluoromethyl, hydroxycarbonyl, aminosulphonyl, C₁-C₄ alkoxy, C₁-C₄ alkoxycarbonyl, C₁-C₆ alkylaminocarbonyl, C₁-C₆ alkylaminosulphonyl, benzylaminosulphonyl, C₃-C₇ cycloalkyl, phenyl, 5- to 10-membered heterocyclyl, 5- to 10-membered heteroaryl and —OR²³,
         wherein phenyl, heterocyclyl and heteroaryl can be substituted with 1 to 3 substituents, whereby the substituents are selected independently of one another from the group consisting of halogen, cyano, nitro, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl, C₁-C₄ alkoxycarbonyl and benzyl,
         and
         wherein alkoxy can be substituted with a substituent selected from the group consisting of halogen, cyano, trifluoromethyl, hydroxy, hydroxycarbonyl, aminocarbonyl, aminosulphonyl, C₁-C₄ alkoxy, C₁-C₄ alkoxycarbonyl, C₁-C₄ alkylaminocarbonyl, C₁-C₄ alkylaminosulphonyl, C₃-C₇ cycloalkyl, phenyl, 5- to 10-membered heterocyclyl and 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, Cᵣ C₄ alkoxy, C₁-C₄ alkylamino, C₁-C₄ alkylsulphonyl and C₁-C₄ alkoxycarbonyl,
         and
         R²³ represents C₃-C₇ cycloalkyl, phenyl, 5- to 10-membered heterocyclyl or 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      and
      whereby
      r represents a number 0, 1, 2 or 3,
      s represents a number 0, 1, 2 or 3,
      t represents a number 2 or 3,
      w represents a number 2 or 3,
      R¹⁹ represents C₁-C₆ alkyl, C₂-C₄ alkenyl, C₁-C₆ alkoxy, phenyl, benzyloxy or 5- to 10-membered heterocyclyl,
         whereby alkyl, alkenyl and alkoxy can be substituted with a substituent, whereby the substituent is selected from the group consisting of halogen, cyano, hydroxy, trifluoromethyl, hydroxycarbonyl, aminosulphonyl, C₁-C₄ alkoxy, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      R²⁰ represents hydrogen, C₁-C₄ alkyl or phenyl,
         whereby alkyl can be substituted with a substituent, whereby the substituent is selected from the group consisting of methoxy, methoxycarbonyl, C₃-C₇ cycloalkyl, phenyl, 5- to 10-membered heterocyclyl and 5- to 10-membered heteroaryl,
         wherein cycloalkyl, phenyl, heterocyclyl and heteroaryl for their part can be substituted with 1 to 3 substituents, whereby the substituents are selected independently of one another from the group consisting of halogen, cyano, hydroxy, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl, C₁-C₄ alkylsulphonylamino and C₁-C₄ alkoxycarbonyl,
      R²¹ represents hydrogen or C₁-C₄ alkyl,
      R²⁸ represents hydrogen or C₁-C₄ alkyl,
      R²⁹ represents C₁-C₆ alkyl, C₂-C₄ alkenyl, C₁-C₆ alkoxy, phenyl, benzyloxy or 5- to 10-membered heterocyclyl,
         whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      R³⁰ represents hydrogen or C₁-C₄ alkyl,
      R³¹ represents C₁-C₆ alkyl, C₂-C₄ alkenyl, phenyl or 5- to 10-membered heterocyclyl,
         whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
R⁸ represents hydrogen, oxo, trifluoromethyl, trifluoromethoxy, C₁-C₄ alkyl, C₁-C₄ alkoxy or C₁-C₄ alkylthio,
R⁹ represents hydrogen, C₁-C₄ alkyl or C₁-C₄ alkoxy,
R¹⁰ represents hydrogen or C₁-C₄ alkyl,
R¹¹ represents hydrogen or C₁-C₄ alkyl,
R¹² represents hydrogen or C₁-C₄ alkyl,
R¹³ represents hydrogen or C₁-C₄ alkyl,
R³ represents hydrogen, halogen, cyano, methyl, methoxy, ethoxy or phenoxy,
R⁴ represents hydrogen, halogen, methyl, methoxy or ethoxy,
R⁵ represents hydrogen, halogen, cyano, nitro, hydroxy, amino, trifluoromethyl, trifluoromethoxy, hydroxycarbonyl, aminocarbonyl, hydroxymethyl, aminomethyl, C₁-C₄ alkyl, C₁-C₄ alkoxy, C₁-C₄ alkylamino, C₁-C₄ alkylthio, C₁-C₄ alkylcarbonyl, C₁-C₄ alkoxycarbonyl, C₁-C₄ alkylaminocarbonyl, C₃-C₆ cycloalkylaminocarbonyl, C₁-C₄ alkylcarbonylamino, C₁-C₄ alkoxycarbonylamino, C₁-C₄ alkylsulphonyl, C₁-C₄ alkylsulphonylamino, C₂-C₄ alkenylsulphonylamino, C₁-C₄ alkylsulphonyl(C₁-C₄ alkyl)amino, benzylsulphonylamino, 5- or 6-membered heteroarylsulphonylamino or 5- to 7-membered heterocyclyl,
   whereby alkylaminocarbonyl, alkylcarbonylamino and alkylsulphonylamino can be substituted with a substituent, whereby the substituent is selected from the group consisting of cyano, hydroxy, amino, hydroxycarbonyl, C₁-C₄ alkoxy, C₁-C₄ alkylamino, morpholinyl, piperidinyl, pyrrolidinyl and benzylamino,
R⁶ represents hydrogen, halogen, C₁-C₄ alkyl or C₁-C₄ alkoxy,
R⁷ represents hydrogen, halogen, C₁-C₄ alkyl or C₁-C₄ alkoxy,
or
R⁵ and R⁶ are bonded to neighbouring carbon atoms and together with the carbon atoms to which they are bonded form a 1,3-dioxolane,
   and their salts, their solvates and the solvates of their salts.

Compounds of the invention are compounds of formula (I) and their salts, their solvates and solvates of their salts; compounds of the formulae named in the following encompassed by formula (I), their salts, solvates and solvates of the as well as those encompassed by formula (I) named in the following as exemplary embodiments and their salts, solvates and solvates of the salts, in so far as that compounds of formula (I) named in the following are not already salts, solvates and solvates of the salts.

The compounds of the invention depending on their structure can exist in stereoisomeric forms (enantiomers, diastereoisomers). The invention therefore comprises the enantiomers or diastereoisomers and their respective mixtures. The stereoisomerically uniform components can be isolated from such mixtures of enantiomers and/or diastereomers by known methods.

Where the compounds of the invention can exist in tautomeric forms, the present invention encompasses all tautomeric forms.

Salts preferred for the purpose of the present invention are physiologically acceptable salts of the compounds of the invention. However, also included are salts which themselves are not suitable for pharmaceutical applications but can be used, for example, for the isolation or purification of the compounds of the invention.

Physiologically acceptable salts of the compounds of the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of common bases such as by way of example and preferably alkali metal salts (e.g. sodium and potassium salts), alkaline earth salts (e.g. calcium and magnesium salts) and ammonium salts, derived from ammonia or organic amines with 1 to 16 C atoms such as by way of example and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates for the purposes of the present invention refer to those forms of the compound which in the solid or liquid state form a complex through coordination with solvent molecules. Hydrates are a special form of solvates with which coordination with water occurs.

For the purposes of the present invention the substituents have the following meaning unless otherwise specified:

Alkyl as well as the alkyl parts in alkoxy, alkylamino, alkylthio, alkylcarbonyl, alkylsulphonyl, alkoxycarbonyl, alkylaminocarbonyl, alkylamino sulphonyl, alkylcarbonylamino, alkoxycarbonyl-amino, alkylsulphonylamino and alkylsulphonyl(C₁-C₄ alkyl)amino represents linear or branched alkyl and unless otherwise stated comprise C₁-C₆ alkyl, particularly C₁-C₄ alkyl, such as by way of example methyl, ethyl, propyl, isopropyl, butyl, isobutyl.

Alkenyl represents a linear or branched alkenyl radical having 2 to 4 carbon atoms. Preferred is a linear alkenyl radical with 2 to 3 carbon atoms. Named by way of example and preferably are: vinyl, allyl, n-prop-1-en-1-yl and n-but-2-en-1-yl.

For the purposes of the invention alkoxy represents a linear or branched alkoxy radical having 1 to 6, 1 to 4 or 1 to 3 carbon atoms. A linear or branched alkoxy radical with 1 to 3 carbon atoms is preferred. Named by way of example and preferably are: methoxy, ethoxy, n-propoxy, isopropoxy, t-butoxy, n-pentoxy and n-hexoxy.

For the purposes of the invention alkylamino represents an amino group having one or two linear or branched alkyl substituents (selected independently of one another) preferably having 1 to 6, 1 to 4 or 1 to 2 carbon atoms. By way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino. C₁-C₃ alkylamino by way of example represents a monoalkylamino radical having 1 to 3 carbon atoms or for a dialkylamino radical having 1 to 3 carbon atoms per alkyl substituent.

Alkylthio by way of example and preferably represents methylthio, ethylthio, n-propylthio, isopropylthio, tert.-butylthio, n-pentylthio and n-hexylthio.

Alkylcarbonyl by way of example and preferably represents methylcarbonyl, ethylcarbonyl, n-propylcarbonyl, iso-propylcarbonyl, n-butylcarbonyl and tert-butylcarbonyl.

Alkylsulphonyl by way of example and preferably represents methylsulphonyl, ethylsulphonyl, n-propylsulphonyl, isopropylsulphonyl, tert.-butylsulphonyl, n-pentylsulphonyl and n-hexylsulphonyl.

Alkoxycarbonyl by way of example and preferably represents methoxycarbonyl, ethoxycarbonyl, n-propoxycarbonyl, isopropoxycarbonyl, t-butoxycarbonyl, n-pentoxycarbonyl and n-hexoxycarbonyl.

For the purposes of the invention alkylaminocarbonyl represents an aminocarbonyl group having one or two linear or branched alkyl substituents (selected independently of one another) preferably having 1 to 6, 1 to 4 or 1 to 2 carbon atoms. By way of example and preferably methylaminocarbonyl, ethylaminocarbonyl, n-propylaminocarbonyl, isopropyl-aminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-tert.-butyl-N-methylaminocarbonyl, N-ethyl-N-n-pentylaminocarbonyl and N-n-hexyl-N-methylaminocarbonyl. C₁-C₃ alkylaminocarbonyl by way of example represents a monoalkylaminocarbonyl radical having 1 to 3 carbon atoms or for a dialkylaminocarbonyl radical having 1 to 3 carbon atoms per alkyl substituent.

For the purposes of the invention alkylaminosulphonyl represents an aminosulphonyl group having one or two linear or branched alkyl substituents (selected independently of one another) preferably having 1 to 6, 1 to 4 or 1 to 2 carbon atoms. By way of example and preferably methylaminosulphonyl, ethylaminosulphonyl, n-propylaminosulphonyl, isopropylaminosulphonyl, tert-butylaminosulphonyl, n-pentylamino sulphonyl, n-hexylaminosulphonyl, N,N-dimethylaminosulphonyl, N,N-diethylaminosulphonyl, N-ethyl-N-methylaminosulphonyl, N-methyl-N-n-propylaminosulphonyl, N-isopropyl-N-n-propylaminosulphonyl, N-tert-butyl-N-methylaminosulphonyl, N-ethyl-N-n-pentylaminosulphonyl and N-n-hexyl-N-methylaminosulphonyl. C₁-C₃ alkylaminosulphonyl represents by way of example represents a monoalkylaminosulphonyl radical having 1 to 3 carbon atoms or for a dialkylaminosulphonyl radical having 1 to 3 carbon atoms per alkyl substituent.

Alkylcarbonylamino by way of example and preferably represents methylcarbonylamino, ethylcarbonylamino, n-propylcarbonylamino, iso-propylcarbonylamino, n-butylcarbonylamino and tert.-butylcarbonylamino.

Alkoxycarbonylamino by way of example and preferably represents methoxycarbonylamino, ethoxycarbonylamino, n-propoxycarbonylamino, isopropoxycarbonylamino, t-butoxycarbonyl-amino, n-pentoxycarbonylamino and n-hexoxycarbonylamino.

Alkylsulphonylamino by way of example and preferably represents methylsulphonylamino, ethylsulphonylamino, n-propylsulphonylamino, isopropylsulphonylamino, tert.-butylsulphonylamino, n-pentylsulphonylamino and n-hexylsulphonylamino.

Alkenylsulphonylamino by way of example and preferably represents vinylsulphonylamino, allylsulphonylamino, n-prop-1-en-1-ylsulphonylamino and n-but-2-en-1-ylsulphonylamino.

Cycloalkyl represents a cycloalkyl group having usually 3 to 7 carbon atoms, by way of example and preferably cyclopropyl, cyclobutyl, cyclopentyl, cyclohexyl and cycloheptyl.

Cycloalkylaminocarbonyl by way of example and preferably represents cyclopropylaminocarbonyl, cyclobutylaminocarbonyl, cyclopentylaminocarbonyl, cyclohexylaminocarbonyl and cycloheptyl-aminocarbonyl.

Heterocyclyl represents a mono- or bicyclic heterocyclic radical having usually 3 to 10, preferably 5 to 8 ring atoms and up to 3, preferably up to 2 heteroatoms and/or hetero-groups from the series N, O, S, SO, SO₂, whereby a nitrogen atom can also form an N-oxide. The heterocyclyl radical can be saturated or partially unsaturated. Preferred are 5- to 8-membered, monocyclic saturated heterocyclyl radicals having up to two heteroatoms from the series O, N and S, by way of example and preferably oxetan-3-yl, pyrrolidin-2-yl, pyrrolidin-3-yl, pyrrolinyl, tetrahydrofuranyl, tetrahydrothienyl, pyranyl, piperidin-1-yl, piperidin-2-yl, piperidin-3-yl, piperidin-4-yl, thiopyranyl, morpholin-1-yl, morpholin-2-yl, morpholin-3-yl, perhydroazepinyl, piperazin-1-yl, piperazin-2-yl.

Heteroaryl represents a 5- to 10-membered aromatic mono- or bicyclic heterocycle, preferably a 5- or 6-membered aromatic monocyclic heterocycle having up to 3 heteroatoms from the series S, O and/or N, whereby the heterocycle can also exist in the form of an N-oxide, for example for indolyl, 1H-indazolyl, 1H-1,2,3-benzotriazolyl, 1H-benzimidazolyl, pyridyl, pyrimidyl, thienyl, furyl, pyrrolyl, thiazolyl, pyrazolyl, thiadiazolyl, N-triazolyl, isoxazolyl, oxazolyl or imidazolyl. Preferred are pyridyl, thienyl, furyl and thiazolyl.

Halogen represents fluorine, chlorine, bromine or iodine, whereby fluorine and chlorine are preferred unless otherwise stated.

The radical definitions given above generally or in preferred ranges definitions apply both to the final products of formula (I) and in each case to the corresponding starting materials and intermediates required for the preparation.

The radical definitions stated individually in the respective combinations and preferred combinations of radicals are also arbitrarily replaced by radical definitions of other combinations, independently of the respective stated combinations of radicals.

The invention also relates preferably to compounds of formula (I) in which

R¹ and R² together with the carbon atom to which they are bonded form a group of formula

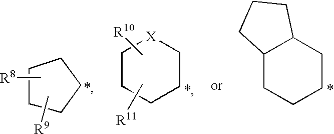

whereby
* represents the carbon atom to which R¹ and R² are bonded,
n represents the number 2,
X represents an oxygen atom, a sulphur atom or NR¹⁴,
   whereby
   R¹⁴ represents C₁-C₆ alkyl, C₂-C₄ alkenyl, C₁-C₄ alkylsulphonyl, benzylsulphonyl, —(CH₂)ₒCOR¹⁶ or —(CH₂)ₚCONR¹⁷R¹⁸,
      whereby alkyl, alkenyl and alkylsulphonyl can be substituted with 1 to 2 substituents, whereby the substituents are selected independently of one another from the group consisting of halogen, cyano, hydroxy, trifluoromethyl, hydroxycarbonyl, aminosulphonyl, C₁-C₄ alkoxy, C₁-C₄ alkoxycarbonyl, C₃-C₇ cycloalkyl, phenyl, 5- to 10-membered heterocyclyl and 5- to 10-membered heteroaryl,
         wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      and
      whereby
      o represents a number 0, 1, 2 or 3,
      p represents a number 0, 1, 2 or 3,
      R¹⁶ represents C₁-C₆ alkyl, C₂-C₄ alkenyl, C₁-C₆ alkoxy, phenyl, benzyloxy or 5- to 10-membered heterocyclyl,
         whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      R¹⁷ represents hydrogen, C₁-C₄ alkyl or phenyl,
         whereby alkyl can be substituted with a substituent, whereby the substituent is selected from the group consisting of methoxy, C₃-C₇ cycloalkyl, phenyl, 5- to 10-membered heterocyclyl and 5- or 6-membered heteroaryl,
         wherein phenyl and heteroaryl for their part can be substituted with 1 to 3 substituents, whereby the substituents are selected independently of one another from the group consisting of halogen, cyano, trifluoromethyl, trifluoromethoxy and C₁-C₄ alkyl,
      R¹⁸ represents hydrogen or C₁-C₄ alkyl,
R⁸ represents hydrogen, oxo, trifluoromethyl, trifluoromethoxy, C₁-C₄ alkyl, C₁-C₄ alkoxy or C₁-C₄ alkylthio,
R⁹ represents hydrogen, C₁-C₄ alkyl or C₁-C₄ alkoxy,
R¹⁰ represents hydrogen,
R¹¹ represents hydrogen,
R³ represents hydrogen, halogen, methyl, methoxy, ethoxy or phenoxy,
R⁴ represents hydrogen, halogen, methyl, methoxy or ethoxy,
R⁵ represents hydrogen, halogen, cyano, nitro, hydroxy, amino, trifluoromethyl, trifluoromethoxy, hydroxycarbonyl, aminocarbonyl, hydroxymethyl, aminomethyl, C₁-C₄ alkyl, C₁-C₄ alkoxy, C₁-C₄ alkylamino, C₁-C₄ alkylthio, C₁-C₄ alkylcarbonyl, C₁-C₄ alkylaminocarbonyl, C₃-C₆ cycloalkylaminocarbonyl, C₁-C₄ alkylcarbonylamino, C₁-C₄ alkoxycarbonylamino, C₁-C₄ alkylsulphonyl, C₁-C₄ alkylsulphonylamino, C₂-C₄ alkenylsulphonylamino, C₁-C₄ alkylsulphonyl(C₁-C₄ alkyl)amino, benzylsulphonylamino, 5- or 6-membered heteroarylsulphonylamino or 5- to 7-membered heterocyclyl,
   whereby alkylaminocarbonyl, alkylcarbonylamino and alkylsulphonylamino can be substituted with a substituent, whereby the substituent is selected from the group consisting of cyano, hydroxy, amino, hydroxycarbonyl, C₁-C₄ alkoxy, C₁-C₄ alkylamino, morpholinyl, piperidinyl, pyrrolidinyl and benzylamino,
R⁶ represents hydrogen, halogen, C₁-C₄ alkyl or C₁-C₄ alkoxy,
R⁷ represents hydrogen,
   or
R⁵ and R⁶ are bonded to neighbouring carbon atoms and together with the carbon atoms to which they are bonded form a 1,3-dioxalane,
   and their salts, their solvates and the solvates of their salts.

The invention also relates preferably to compounds of formula (I) in which
R¹ and R² together with the carbon atom to which they are bonded form a group of the formula

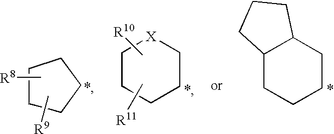

whereby
* represents the carbon atom to which R¹ and R² are bonded,
n represents the number 2,
X represents NR¹⁴,
   whereby
   R¹⁴ represents C₁-C₄ alkyl, C₂-C₄ alkenyl, benzylsulphonyl, —(CH₂)ₒCOR¹⁶ or —(CH₂)ₚCONR¹⁷R¹⁸,
      whereby alkyl and alkenyl can be substituted with 1 to 2 substituents, whereby the substituents are selected independently of one another from the group consisting of halogen, cyano, hydroxy, trifluoromethyl, hydroxycarbonyl, aminosulphonyl, C₁-C₄ alkoxy, C₁-C₄ alkoxycarbonyl, C₃-C₇ cycloalkyl, phenyl, 5- to 10-membered heterocyclyl and 5- to 10-membered heteroaryl,
         wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
      and
      whereby
      o represents a number 1 or 2,
      p represents a number 1 or 2,
      R¹⁶ represents C₁-C₄ alkyl, C₁-C₄ alkoxy, phenyl or benzyloxy,
      R¹⁷ represents hydrogen, C₁-C₄ alkyl or phenyl,
         whereby alkyl can be substituted with a substituent, whereby the substituent is selected from the group consisting of methoxy, phenyl and 5- or 6-membered heteroaryl,
         wherein phenyl for its part can be substituted with 1 to 3 substituents, whereby the substituents are selected independently of one another from the group consisting of halogen, cyano, trifluoromethyl, trifluoromethoxy and C₁-C₄ alkyl,
      R¹⁸ represents hydrogen,
R⁸ represents hydrogen, C₁-C₄ alkyl or C₁-C₄ alkoxy,
R⁹ represents hydrogen or C₁-C₄ alkyl,
R¹⁰ represents hydrogen,
R¹¹ represents hydrogen,
R³ represents hydrogen, halogen, methyl, ethoxy or phenoxy,
R⁴ represents hydrogen, halogen or methyl,
R⁵ represents hydrogen, halogen, cyano, hydroxy, trifluoromethyl, trifluoromethoxy, hydroxycarbonyl, aminocarbonyl, hydroxymethyl, C₁-C₄ alkyl, C₁-C₄ alkoxy, C₁-C₄ alkylaminocarbonyl, C₃-C₆ cycloalkylaminocarbonyl, C₁-C₄ alkylcarbonylamino, C₁-C₄ alkoxycarbonylamino, C₁-C₄ alkylsulphonyl, C₁-C₄ alkylsulphonylamino, C₂-C₄ alkenylsulphonylamino, C₁-C₄ alkylsulphonyl(C₁-C₄ alkyl)amino, benzylsulphonylamino or 5- or 6-membered heteroarylsulphonylamino,
   whereby alkylaminocarbonyl, alkylcarbonylamino and alkylsulphonylamino can be substituted with a substituent, whereby the substituent is selected from the group consisting of amino, C₁-C₄ alkylamino, morpholinyl and pyrrolidinyl,
R⁶ represents hydrogen, halogen, C₁-C₄ alkyl or C₁-C₄ alkoxy,
R⁷ represents hydrogen,
   and their salts, their solvated and the solvates of their salts.

The invention also relates preferably to compounds of formula (I) in which R¹ and R² together with the carbon atom to which they are bonded form a group of the formula

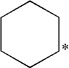

whereby * represents the carbon atom to which R¹ and R² are bonded.

The invention also relates preferably to compounds of formula (I) in which R³ represents hydrogen, halogen or methyl.

The invention also relates preferably to compounds of formula (I) in which R⁴ represents hydrogen, halogen or methyl.

The invention also relates preferably to compounds of formula (I) in which R⁵ represents hydroxy, amino, hydroxycarbonyl, aminocarbonyl, hydroxymethyl, aminomethyl, C₁-C₄ alkylamino, C₁-C₄ alkylcarbonyl, C₁-C₄ alkylaminocarbonyl, C₃-C₆ cycloalkylaminocarbonyl, C₁-C₄— alkylcarbonylamino, C₁-C₄ alkoxycarbonylamino, C₁-C₄ alkylsulphonylamino, C₂-C₄ alkenylsulphonylamino, C₁-C₄ alkylsulphonyl(C₁-C₄ alkyl)amino, benzylsulphonylamino, 5- or 6-membered heteroarylsulphonylamino or 5- to 7-membered heterocyclyl,
whereby alkylaminocarbonyl, alkylcarbonylamino and alkylsulphonylamino can be substituted with a substituent, whereby the substituent is selected from the group consisting of cyano, hydroxy, amino, hydroxycarbonyl, C₁-C₄ alkoxy, C₁-C₄ alkylamino, morpholinyl, piperidinyl, pyrrolidinyl and benzylamino.

The invention also relates preferably to compounds of formula (I) in which R⁵ represents C₁-C₄ alkylcarbonylamino or C₁-C alkylsulphonylamino,
whereby alkylcarbonylamino and alkylsulphonylamino can be substituted with a substituent, whereby the substituent is selected from the group consisting of cyano, hydroxy, amino, hydroxycarbonyl, C₁-C₄ alkoxy, C₁-C₄ alkylamino, morpholinyl, piperidinyl, pyrrolidinyl and benzylamino.

The invention also relates preferably to compounds of formula (I) in which R⁵ represents C₁-C₄ alkylsulphonylamino,
whereby alkylsulphonylamino can be substituted with a substituent, whereby the substituent is selected from the group consisting of cyano, hydroxy, amino, hydroxycarbonyl, C₁-C₄ alkoxy, C₁-C₄ alkylamino, morpholinyl, piperidinyl, pyrrolidinyl and benzylamino.

The invention also relates preferably to compounds of formula (I) in which R⁵ represents C₁-C₄ alkylsulphonylamino,
whereby alkylsulphonylamino can be substituted with a substituent, whereby the substituent is selected from the group amino, C₁-C₄ alkylamino, morpholinyl and pyrrolidinyl.

The invention also relates preferably to compounds of formula (I) in which R⁵ represents C₁-C₄ alkylsulphonyl.

The invention also relates preferably to compounds of formula (I) in which R⁶ and R⁷ stand for hydrogen.

The invention further relates to a method for the preparation of compounds of formula (I), whereby according to method

[A] compounds of formula

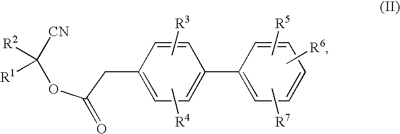

in which
R¹, R², R³, R⁴, R⁵, R⁶ and R⁷ have the meaning indicated above,
are reacted with bases,
or
[B] compounds of formula

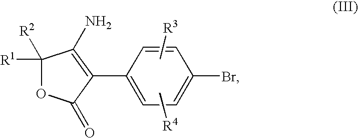

in which
R¹, R², R³ and R⁴ have the meaning indicated above
are reacted under Suzuki coupling conditions with compounds of formula

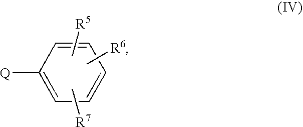

in which
R⁵, R⁶ and R⁷ have the meaning indicated above, and
Q represents —B(OH)₂, a boronic acid ester, preferably a boronic acid pinacol ester, or —BF₃⁻K⁺.

If compounds with free amino functions are formed in the reactions according to method [A] or method [B], these amino functions can be reacted with carboxylic acids, acyl chlorides, alkyl halides, benzyl halides or sulphonyl chlorides by methods known to the skilled person and thus further compounds of formula (I) are formed.

The reaction according to Method [A] is normally carried out in an inert solvent, preferably in a temperature range from room temperature to the reflux of the solvent under atmospheric pressure.

Inert solvents are, for example, hydrocarbons such a toluene or benzene, or other solvents such as dioxan, dimethylformamide, acetonitrile or dichloromethane. It is also possible to use mixtures of the solvents. Dimethylformamide or dichloromethane are particularly preferred.

Bases are, for example, potassium tert.-butylate, sodium hydride, lithium diisopropylamide, sodium, potassium or lithium hexamethyldisilylamide or 2-tert-butylimino-2-diethylamino-1,3-dimethylperhydro-1,3,2-diazaphosphorine (BEMP). Potassium tert.-butylate or 2-tert-butylimino-2-diethylamino-1,3-dimethylperhydro-1,3,2-diazaphosphorine (BEMP) are particularly preferred.

The reaction according to method [B] is normally carried out in inert solvents in the presence of a catalyst, optionally in the presence of an auxiliary, preferably in a temperature range from room temperature to 130° C. under atmospheric pressure.

Catalysts are, for example, the palladium catalysts normal for Suzuki reaction conditions. Catalysts such as, for example, dichlorobis(triphenylphosphine) palladium, tetrakistriphenylphosphine palladium(0), palladium(II) acetate, palladium(II) acetate/triscyclohexylphosphine or bis-(diphenylphosphaneferrocenyl)palladium(II) chloride or palladium(II) acetate with a ligand such as dicyclohexyl-(2′,4′,6′-triisopropyl-biphenyl-2-yl)phosphine are preferred.

Auxiliaries are, for example, potassium acetate, caesium, potassium or sodium carbonate, potassium tert.-butylate, caesium fluoride or potassium phosphate. Auxiliaries such as, for example, potassium acetate and/or aqueous sodium carbonate solution are preferred.

Inert solvents are, for example, ethers such as dioxan, tetrahydrofuran or 1,2-dimethoxyethane, hydrocarbons such as benzene, xylene or toluene, or carboxamides such as dimethylformamide or dimethylacetamide, alkylsulphoxides such as dimethylsulphoxide, or N-methylpyrrolidone, or mixtures of the solvents with an alcohol such as methanol or ethanol and/or water, 1,2-Dimethoxyethane or a mixture of 1,2-dimethoxyethane with ethanol and water is preferred.

Compounds of the formula (III) may be synthesized by Method [A] from the corresponding starting materials.

Compounds of formula (IV) are known or may be synthesized by known methods from the corresponding starting materials.

Compounds of formula (II) are known or can be prepared by reacting compounds of formula

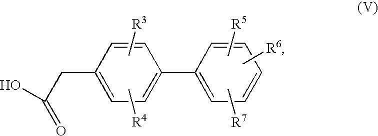

in which
R³, R⁴, R⁵, R⁶ and R⁷ have the meaning indicated above,
in the first stage with thionyl chloride or oxalyl chloride and in the second stage with a compound of the formula

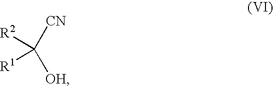

in which
R¹ and R² have the meaning defined above.

The reaction of the compound of the formula (V) with thionyl chloride or oxalyl chloride in the first stage is normally carried out in an inert solvent, preferably in a temperature range from room temperature to the reflux of the solvent under atmospheric pressure.

Inert solvents are, for example, halogenated hydrocarbons such as dichloromethane or dichloroethane, hydrocarbons such as benzene, xylene or toluene, or other solvents such as chlorobenzene, toluene is preferred.

The reaction of the acid chloride with a compound of the general formula (VI) in the second stage is usually carried out in inert solvents, preferably in a temperature range of 50° C. to the reflux of the solvent under atmospheric pressure.

Inert solvents are, for example, hydrocarbons such as benzene, xylene or toluene, or other solvents such as chlorobenzene, toluene is preferred.

The compounds of formulae (V) and (VI) are known or may be synthesized from the corresponding starting materials by known methods.

In an alternative method the reaction of the compounds of the formula (V) with compounds of the formula (VI) can also be carried out via the thiocarbonic esters of the compounds of formula (V) in the presence of bases, for example dimethylaminopyridine.

The preparation of the compounds of the invention can be illustrated by the following synthesis scheme.

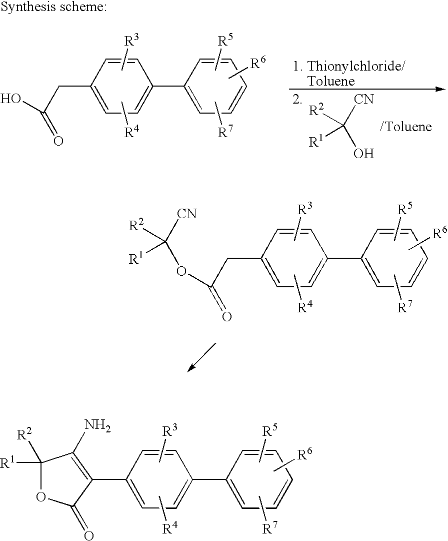

The compounds of the invention show an unexpected, valuable spectrum of pharmacological activity.

They are therefore suitable for use as medicament in the treatment and/or prophylaxis of diseases in humans and animals.

The compounds of the present invention are characterized in particular by an advantageous anti-retroviral activity spectrum.

The present invention relates further to the use of the compounds of the invention for the treatment and/or prophylaxis of diseases that are caused by retroviruses, in particular HI viruses.

The present invention relates further to the use of the compounds of the invention for the treatment and/or prophylaxis of diseases, in particular the previously indicated diseases.

The present invention relates further to the use of the compounds of the invention for the manufacture of a medicament for the treatment and/or prophylaxis of diseases, in particular the previously indicated diseases.

The present invention relates further to a method for the treatment and/or prophylaxis of diseases, in particular the previously named diseases, by the use of a therapeutically effective amount of the compounds of the invention.

Therapeutic indications in human medicine which may be mentioned by way of example are:
1.) The treatment and prophylaxis of human retrovirus infections.
2.) For the treatment and prophylaxis of HIV I (virus of human immune deficiency; previously called HTLV III/LAV) and HIV II-induced infections and diseases (AIDS) and the stages associated therewith such as ARC (AIDS related complex) and LAS (lymphadenophathy syndrome) the immune deficiency and encephalopathy caused by this virus.
3.) For the treatment of HIV infections caused by single, multiple or multiresistant HIV viruses.

Resistant HI viruses means for example, viruses with resistance towards nucleosidic inhibitors (RTI), non-nucleosidic inhibitors (NNRTI) or protease inhibitors (PI) or viruses with resistance towards other activity principles, e.g. T20 (fusion inhibitors).
4.) For the treatment or prophylaxis of AIDS carrier states.
5.) For the treatment or prophylaxis of an HTLV-I or HTLV-II infection

Indication in veterinary medicine which may be mentioned by way of example are:

Infections with
a) Maedivisna (in sheep and goats)
b) progressive pneumonia virus (PPV) (in sheep and goats)
c) caprine arthritis encephalitis virus (in sheep and goats)
d) Zwoegerziekte virus (in sheep)
e) infectious anaemia virus (of the horse)
f) infections caused by the feline leukaemia virus
g) infection caused by the feline immune deficiency virus (FIV)
h) infection caused by the simian immune deficiency virus (SIV)

The therapeutic indications listed above under points 2, 3 and 4 are preferred.

The present invention relates further to medicaments comprising of at least one compound of the invention and at least one or more active substances, in particular for the treatment and/or prophylaxis of the previously named diseases.

The compounds of the invention can also be used advantageously, particularly in the points 2, 3 and 4 listed above, as components of a combination therapy with one or more other compounds active in these therapeutic areas. For example, the compounds can be used in combination with effective doses of antiviral substances which are based on the activity principles listed below:

HIV protease inhibitors; named by way of example: saquinavir, indinavir, ritonavir, nelfinavir, amprenavir, tipranavir;
Nucleosidic and non-nucleosidic inhibitors of HIV reverse transcriptase; named by way of example: zidovudin, lamivudin, didanosin, zalzitabin, stavudin, abacavir, tenofovir, adefovir, nevirapin, delavirdin, efavirenz, emtricitabin, etravirin, rilpivirin;
HIV integrase inhibitors, named by way of example: S1360, L870810;
HIV fusion inhibitors; named by way of example: pentafuside, T1249.

Cytochrome P450 monooxygenase inhibitors; named by way of example: ritonavir.

This selection is to illustrate the combination possibilities, not, however, to serve to restrict to the examples listed here; in principle every combination of the compounds of the invention with antivirally active substances is to be considered within the scope of the invention.

The compounds of the invention can act systemically and/or locally. For this purpose they can be applied in a suitable way, for example, orally, parenterally, pulmonally, nasaly, sublingually, lingually, buccally, rectally, dermally, transdermally, conjunctivally, optically or as implant or stent.

The compounds of the invention can be administered in suitable dosage forms for these administration routes.

Suitable for oral administration are administration forms which contain the compounds of the invention in crystalline and/or amorphous and/or in dissolved form and which function according to the prior art to release the compounds of the invention rapidly or in modified form, e.g. tablets (uncoated or coated tablets, for example having coatings which are resistant to gastric juices or dissolve with delay or are insoluble which control the release of the compounds of the invention), tablets or films/wafers which disintegrate rapidly in the oral cavity, or films/wafers, films/lyophylisates, capsules (for example hard or soft gelatine capsules), film-coated tablets, granules, pellets, powders, emulsions, suspensions, aerosols or solutions.

Parenteral administration can take place with avoidance of an absorption step (e.g. intravenously, intraarterielly, intracardially, intraspinally or intralumbally) or with involvement of absorption (e.g. intramuscularly, subcutaneously, intracutaneously, percutaneously or intraperitonealy). Administration forms suitable for parenteral administration are i.a. injection and infusion preparations in the form of solutions, suspensions, emulsions, lyophylisates or sterile powders.

Suitable for other administration routes are, for example, medication forms for inhalation (i.a. powder inhalators, nebulizers), nasal drops, solutions, sprays; tablets administered lingually, sublingually or buccally, films/wafers or capsules, suppositories, preparations for ears or eyes, vaginal capsules, aqueous suspensions (lotions, shaking mixtures), lipophilic suspensions, ointments, creams, transdermal therapeutic systems (for example, plasters), milk, pastes, foams, dusting powders, implants or stents.

The compounds of the invention can be transformed into the stated administration forms. This can be carried out in a known way by mixing with inert, non-toxic, pharmaceutically acceptable excipients. These excipients include i.a. carriers (e.g. microcrystalline cellulose, lactose, mannitol), solvents (e.g. liquid polyethyleneglycols), emulsifiers and dispersants or wetting agents (for example, sodium dodecyl sulphate, polyoxysorbitanoleate), binding agents (for example polyvinylpyrrolidone), synthetic and natural polymers (for example, albumin), stabilizers (e.g. antioxidants such as for example ascorbic acid), colours (e.g. inorganic pigments for example iron oxides) and taste and/or odour corrigents.

The present invention relates further to medicaments comprising at least one compound of the invention, usually together with one or more inert, non-toxic, pharmaceutically acceptable excipients, and their use for the previously described purposes.

In general it has proved to be advantageous in both human and veterinary medicine to dose the active compound(s) of the invention in total amounts of 0.1 to 200 mg/kg, preferably 1 to 100 mg/kg body weight every 24 hours, where appropriate in the form of several individual doses to achieve the desired results. A single dose contains the active compound(s) in amounts of 1 to 80 mg/kg, in particular 1 to 30 mg/kg body weight.

Nevertheless, it may be necessary where appropriate to deviate from the said amounts, in particular depending upon body weight, administration route, individual behaviour towards the active ingredient, type of preparation and time point or interval at which administration is carried out. Thus in a few cases it may be sufficient to used less than the aforementioned minimum amount, whereas in other cases the named upper limit must be exceeded. In the instances of an administration of larger amounts it may be advisable to divide these into several individual doses throughout the day.

The percentage amounts in the following tests and examples, unless otherwise stated, are percentages by weight; parts are parts by weight. Solvent ratios and concentration amounts of liquid/liquid solutions relate in each case to volume. The statement “w/v” means “weight/volume”. Thus, for example “10% w/v”: 100 ml solution or suspension contains 10 g substance.

### A) EXAMPLES

### Abbreviations

GWP general working procedure
aq. aqueous, aqueous solution
DIC direct chemical ionization (in MS)
DCM dichloromethane
DIPPEA diisopropylethylamine
DMA N,N-dimethylacetamide
DME dimethoxyethane
DMF N,N-dimethylformamide
DMSO dimethylsulphoxide
th. of theory (with yields)
EDC N′-(3-dimethylaminopropyl)-N-ethylcarbodiimide x HCl
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high pressure-, high performance liquid chromatography
conc. concentrated
LC-MS liquid chromatography-couple mass spectrometry
min minute(s)
MS mass spectrometry
NMR nuclear magnetic resonance
PyBOP benzotriazol-1-yloxy-tris(pyrrolidino)phosphonium hexafluorophosphate
Rₜ retention time (in HPLC)
RT room temperature
TBTU O-(benzotriazol-1-yl)-N,N,N′,N′-tetramethyluronium tetrafluoroborate
TFA trifluoroacetic acid
THF tetrahydrofuran
TMOF trimethylorthoformate

### LC-MS and HPLC Methods:

Method 1 (LC-MS): Instrument: Micromass Quattro LCZ with HPLC Agilent Series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min 2 ml/min; oven: 50° C.; UV detection: 208-400 nm.

Method 2 (LC-MS): Instrument MS: Micromass ZQ; instrument HPLC: Waters Alliance 2795; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min 2 ml/min; oven: 50° C.; UV detection: 210 nm.

Method 3 (LC-MS): Instrument MS: Micromass ZQ; instrument HPLC: HP 1100 Series; UV DAD; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min. 2 ml/min; oven: 50° C.; UV detection: 210 nm.

Method 4 (LC-MS): Instrument: Micromass Platform LCZ with HPLC Agilent Serie 1100; column: Thermo Hypersil GOLD 3μ 20 mm×4 mm; eluent A: 1 l water+0.5 ml 50% formic acid, eluent B: 1 l acetonitrile+0.5 ml 50% formic acid; gradient: 0.0 min 100% A→0.2 min 100% A→2.9 min 30% A→3.1 min 10% →5.5 min 10% A; oven: 50° C.; flow: 0.8 ml/min; UV detection: 210 nm.

Method 5 (GC-MS): Instrument: Micromass GCT, GC6890; column: Restek RTX-35MS, 30 m×250 μm×0.25 μm; constant flow with helium: 0.88 ml/min; oven: 60° C.; inlet: 250° C.; gradient: 60° C. (hold 0.30 min), 50° C./min→120° C., 16° C./min→250° C., 30° C./min→300° C. (hold 1.7 min).

Method 6 (preparative RP-HPLC): column: Grom-Sil 120 ODS-4HE, 10 SNo. 3355, 250 mm×30 mm. Eluent A: water+0.1% hydrochloric acid, eluent B: acetonitrile. Flow: 50 ml/min. programme: 0-4 min: 10% B; 4.01-33 min: gradient to 90% B.

Method 7 (preparative RP-HPLC): column: Grom-Sil C18, 10 μm, 250 mm×30 mm. Eluent A: water+0.1% formic acid, eluent B: acetonitrile. Flow: 50 ml/min. Programme: 0-5 min: 10% B; 5-38 min: gradient to 95% B.

Method 8 (LC-MS): Instrument: Micromass Quattro LCZ with HPLC Agilent Series 1100; column: Phenomenex Onyx Monolithic C18, 100 mm×3 mm. Eluent A: 1 l water+0.5 ml 50% formic acid, eluent B: 1 l acetonitrile+0.5 ml 50% formic acid; gradient: 0.0 min 90% A→2 min 65% A→4.5 min 5% A→6 min 5% A; flow: 2 ml/min; oven: 40° C.; UV detection: 208-400 nm.

Method 9 (LC-MS): Instrument MS: Micromass ZQ; instrument HPLC: HP 1100 Series; UV DAD; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min. 2 ml/min; oven: 50° C.; UV detection: 210 nm.

Method 10 (Enantiomer separation): Column: Chiralpak IA, 250 mm×21 mm, methyl-tert-butyl ether/methanol 9:1 (vol/vol), flow: 15 ml/min, oven: 30° C., UV detection: 220 nm; analysis: Chiralpak IA, methyl-tert.-butyl ether/methanol 9:1 (vol/vol), flow: 1 ml/min, oven: 25° C., UV detection: 220 nm.

Method 11 (LC-MS): Instrument MS: Micromass TOF (LCT); instrument HPLC: Waters 2690; autosampler: Waters 2700, column: YMC-ODS-AQ, 50 mm×4.6 mm, 3.0 μm; eluent A: water+0.1% formic acid, eluent B: acetonitrile+0.1% formic acid; gradient: 0.0 min 100% A→0.2 min 95% A→1.8 min 25% A→1.9 min 10% A→2.0 min 5% A→3.2 min 5% A→3.21 min 100% A→3.35 min 100% A; oven: 40° C.; flow: 3.0 ml/min; UV detection: 210 nm.

Method 12 (LC-MS): Instrument MS: Waters ZQ 2000; instrument HPLC: Agilent 1100, 2-column switching, autosampler: HTC PAL; column: YMC-ODS-AQ, 50 mm×4.6 mm, 3.0 μm; eluent A: water+0.1% formic acid, eluent B: acetonitrile+0.1% formic acid; gradient: 0.0 min 100% A→0.2 min 95% A→1.8 min 25% A→1.9 min 10% A→2.0 min 5% A→3.2 min 5% A→3.21 min 100% A→3.35 min 100% A; oven: 40° C.; flow: 3.0 ml/min; UV detection: 210 nm.

Method 13 (LC-MS): Instrument MS: Micromass TOF (LCT); instrument HPLC: Waters 2690; autosampler: Waters 2700; column: YMC-ODS-AQ, 50 mm×4.6 mm, 3.0 μm; eluent A: water+0.1% formic acid, eluent B: acetonitrile+0.1% formic acid; gradient: 0.0 min 100% A→0.1 min 95% A→0.8 min 25% A→0.9 min 5% A→1.8 min 5% A→1.81 min 100% A→2.1 min 100% A; oven: 40° C.; flow: 3.0 ml/min; UV detection: 210 nm.

### Starting Compounds

### Example 1A

### 4-Bromo-5-chloro-2-methylaniline

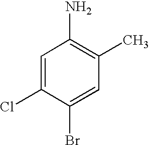

321.6 g (2.01 mol) bromine are added dropwise to 300.0 g (2.12 mol) 5-chloro-2-methylaniline in 6000 ml glacial acetic acid at 10-15° C. When the addition is complete, the reaction is stirred overnight at room temperature, the precipitate is separated by filtration and the filter residue is washed with ethyl acetate. The solid is stirred then with ethyl acetate and saturated sodium hydrogen carbonate solution, the organic phase is separated and the solvent is removed completely with a rotary evaporator. After recrystallization twice from n-hexane 186 g (38% th.) product are obtained.

LC-MS (method 1): Rₜ=2.26 min, m/z=220 (M+H)⁺

### Example 2A

### 1-Bromo-2-chloro-5-methyl-4-(2,2,2-trichloroethyl)benzene

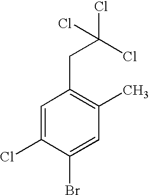

136.1 g (1.01 mol) copper(II) chloride and 1014 ml (1226.7 g, 12.65 mol) dichloroethene are added to 170 ml (148.2 g, 1.27 mol) isopentylnitrite in 554 ml acetonitrile and the suspension is treated portionwise at 30° C. with 186.0 g (0.76 mol) 4-bromo-5-chloro-2-methylaniline. Stirring is continued at room temperature until no further gas generation is recognizable. The reaction mixture is stirred vigorously with 6000 ml 20% hydrochloric acid for 10 min and then extracted several times with tert.-butylmethyl ether, the combined organic phases are washed with 20% hydrochloric acid, dried over sodium sulphate, filtered and the solvent is removed with a rotary evaporator. After purification of the residue over silica gel 60 by low pressure column chromatography (eluent: cyclohexane) 235 g (83% th.) product are obtained. The crude product is reacted further without additional purification.

### Example 3A

### Ethyl 4-bromo-5-chloro-2-methylbenzoate

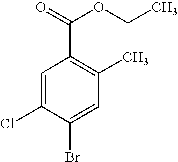

391.4 g (6.98 mol) potassium hydroxide in 3000 ml methanol are treated at room temperature with 235.0 g (0.70 mol) 1-bromo-2-chloro-5-methyl-4-(2,2,2-trichlorethyl)benzene. The mixture is stirred at reflux for 4 hours. For work-up the solution is cooled to room temperature and carefully acidified with concentrated sulphuric acid. The resulting suspension is cooled, the salts are separated by filtration and the mother liquor is evaporated to dryness with a rotary evaporator. The crude material obtained (200 g, quant.) is reacted further without additional purification.

### Example 4A

### (4-Bromo-5-chloro-2-methylphenyl)acetic acid

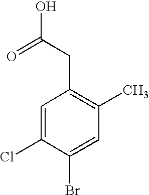

200 g (0.72 mol) ethyl 4-bromo-5-chloro-2-methylbenzoate are dissolved in 1428 ml tetrahydrofuran and treated with a solution of 20.7 g (0.86 mol) lithium hydroxide in 1428 ml water. The reaction is brought to completion overnight at room temperature. For work-up the solvent is removed completely with a rotary evaporator, the residue is treated with water, extracted several times with tert.-butyl methyl ether and the aqueous phase is acidified with concentrated hydrochloric acid. The precipitated crystals are separated by filtration, the filter residue is washed with n-heptane and after drying the solid in a high vacuum 141.5 g (75% th.) product are obtained.

LC-MS (Method 2): Rₜ=2.00 min, m/z=261 (M−H)⁻

¹H NMR (300 MHz, CDCl₃), δ=7.46 (s, 1H), 7.29 (s, 1H), 3.61 (s, 2H), 2.28 (s, 3H).

### Example 5A

### 1-Cyanocyclohexyl (4-bromo-5-chloro-2-methylphenyl)acetate

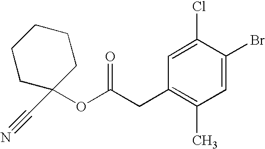

5.3 g (19.97 mmol) (4-bromo-5-chloro-2-methylphenyl)acetic acid are dissolved in 40 ml toluene, treated with 8.7 ml oxalyl chloride, heated to 80° C. and the solution is stirred at this temperature for about 1 hour until no further gas generation is visible. The toluene is removed completely with a rotary evaporator and the residue is dried for ca. 1 hour in a high vacuum. 5.0 g (39.95 mmol) cyclohexanone cyanhydrin is added to the residue and the mixture is stirred overnight 120° C. The cooled crude product is diluted with acetonitrile and purified by RP-HPLC (Method 6). 6.04 g (82% th.) of the desired product are obtained.

LC-MS (Method 1): Rₜ=3.07 min, m/z=370 (M−H)⁻

¹H NMR (400 MHz, DMSO-d₆), δ=7.64 (s, 1H), 7.54 (s, 1H), 3.85 (s, 2H), 2.25-2.08 (m, 5H including: 2.01 (s, 3H)), 1.90 (m, 2H), 1.59 (m, 2H), 1.48 (m, 3H), 1.35 (m, 1H).

### Example 6A

### 4-Amino-3-(4-bromo-5-chloro-2-methylphenyl)-1-oxaspiro[4.5]dec-3-en-2-one

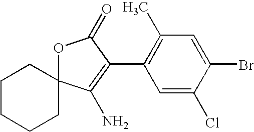

6.0 g (16.29 mmol) 1-cyanocyclohexyl (4-bromo-5-chloro-2-methylphenyl)acetate and 11.2 g (40.74 mmol) 2-tert-butylimino-2-diethylamino-dimethylperhydro-1,2,3-diazaphosphorine are dissolved in 160 ml dichloromethane. The solution is stirred overnight at room temperature, the separated precipitate is filtered off, the filter residue is washed with dichloromethane, the filtrate is evaporated to dryness with a rotary evaporator and the residue is dried in high vacuum. 4.1 g (68% th.) of the desired product are obtained.

LC-MS (Method 3): Rₜ=2.50 min, m/z=370 (M+H)⁺

¹H NMR (400 MHz, DMSO-d₆), δ=7.67 (s, 1H), 7.28 (s, 1H), 7.05 (bs, 2H), 2.12 (s, 3H), 1.98-1.82 (m, 2H), 1.78-1.48 (m, 7H), 1.25 (m, 1H).

### Example 7A

### Benzyl 3-[2-(4-bromo-5-chloro-2-methylphenyl)acetoxy]-3-cyanopiperidine-1-carboxylate

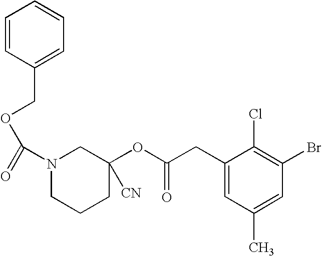

2.89 g (10.95 mmol) (4-bromo-5-chloro-2-methylphenyl)acetic acid are suspended in 150 ml toluene, treated with 3.39 g (14.6 mmol) thiocarbonic acid o,o-di-(2-pyridyl ester), 0.09 g (0.73 mmol) DMAP and 2 g (7.30 mmol) benzyl 3-cyano-3-hydroxypiperidine-1-carboxylate (synthesis from the commercially available ketone by analogy to Swain, Christopher J.; Kneen, Clare; Baker, Raymond, Tetrahedron Letters 1990, 31, 2445) and stirred overnight at 80° C. For work-up the reaction solution is treated with dichloromethane and washed with 1N hydrochloric acid, 1N sodium hydroxide solution and saturated sodium chloride solution. After drying (sodium sulphate) and evaporation in vacuum the residue is purified by preparative RP-HPLC. 2.53 g (68% th.) product are obtained.

LC-MS (Method 1): Rₜ=3.06 min, m/z=505.3 (M+H)⁺

### Beispiel 8A

### Benzyl 4-amino-3-(4-bromo-5-chloro-2-methylphenyl)-2-oxo-1-oxa-7-azaspiro[4.5]dec-3-ene-7-carboxylate

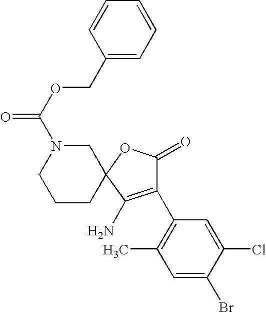

2.5 g (4.94 mmol) benzyl 3-[2-(4-bromo-5-chloro-2-methylphenyl)acetoxy]-3-cyanopiperidine-1-carboxylate and 3.39 g (12.36 mmol) 2-tert.-butylimino-2-diethylamino-1,3-dimethylperhydro-1,3,2-diazaphosphorine are dissolved in 30 ml dichloromethane and stirred overnight at room temperature. The volatile components are removed in vacuum. The residue is recrystallized from dichloromethane/diethyl ether. 2.39 g (89% th.) of the product are obtained.

LC-MS (Method 1): Rₜ=2.55 min, m/z=505.3 (M+H)⁺

### Example 9A

### 4-Amino-3-(4-bromo-5-chloro-2-methylphenyl)-1-oxa-7-azaspiro[4.5]dec-3-en-2-one

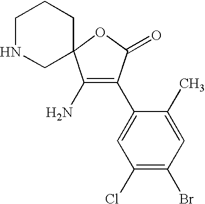

2 g (3.64 mmol) benzyl 4-amino-3-(4-bromo-5-chloro-2-methylphenyl)-2-oxo-1-oxa-7-azaspiro[4.5]-dec-3-ene-7-carboxylate are taken up into 22.7 ml trifluoroacetic acid and stirred overnight at room temperature. For work-up the volatile components are removed in vacuum and the product is purified by RP-HPLC. 1.05 g (77% th.) product are obtained.

LC-MS (Method 3): Rₜ=1.50 min, m/z=371.2 (M+H)⁺

### Beispiel 10A

### 2-[4-Amino-3-(4-bromo-5-chloro-2-methylphenyl)-2-oxo-1-oxa-7-azaspiro[4.5]dec-3-en-7-yl]-N-benzylacetamide

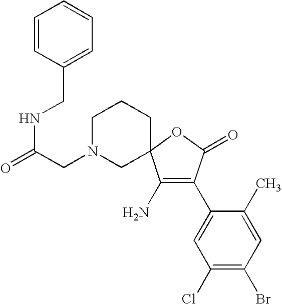

1 g (2.69 mmol) 4-amino-3-(4-bromo-5-chloro-2-methylphenyl)-1-oxa-7-azaspiro[4.5]dec-3-en-2-one is dissolved in 7.2 ml DMF and treated with 0.68 g (2.96 mmol) N-benzyl-2-bromoacetamide and 1.16 g (8.07 mmol) potassium carbonate. Stirring is continued for 10 min at room temperature and then overnight at 50° C. For work-up the reaction mixture is diluted with water, acidified with 1N hydrochloric acid and extracted with dichloromethane. The combined organic phases are dried (sodium sulphate) and evaporated in vacuum. After purification by preparative RP-HPLC 0.96 g (66% th.) of product is obtained.

LC-MS (Method 2): Rₜ=1.88 min, m/z=518.1 (M+H)⁺

### Example 11A

### N-[4′-(4-Amino-2-oxo-1-oxaspiro[4.5]dec-3-en-3-yl)-2′-chloro-5′-methylbiphenyl-3-yl]-3-bromopropane-1-sulphonamide

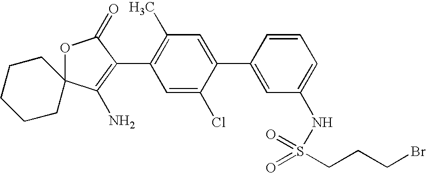

100 mg (0.26 mmol) 4-amino-3-(3′-amino-2-chloro-5-methylbiphenyl-4-yl)-1-oxaspiro[4.5]dec-3-en-2-one are dissolved in 5 ml THF. 63 μl pyridine and 87 mg (0.39 mmol) 3-bromopropyl-sulphonyl chloride are then added and stirring is continued for 18 h at RT. After decanting from the crystals the solution is evaporated in vacuum (130 mg oil). The crude product (130 mg oil, 81% content by LC-MS) contains as by-product ca. 16% N-[4′-(4-amino-2-oxo-1-oxaspiro[4.5] dec-3-en-3-yl)-2′-chloro-5′-methylbiphenyl-3-yl]-3-chloropropane-1-sulphonamide and is reacted without further purification.

LC-MS (Method 1): Rₜ=2.37 min, m/z=567 (M+H)⁺

### Example 12A

### 4-Amino-3-[2-chloro-5-methyl-3′-(methylsulphonyl)biphenyl-4-yl]-1-oxa-7-azaspiro[4.5]dec-3-en-2-one

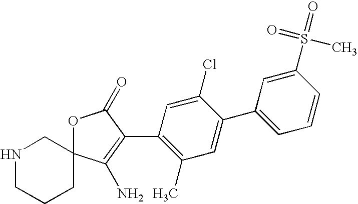

By analogy to the method for Example 9A the title compound is prepared from 5.6 g of the compound from Example 13. Purification by silica gel chromatography, eluent dichloromethane, dichloromethane/methanol (50:1). 4.52 g (quantitative) of the product is obtained as solid.

LC-MS (Method 2): Rₜ=1.28 min, m/z=447 (M+H)⁺

### Example 13A

### {4-Amino-3-[2-chloro-5-methyl-3′-(methylsulphonyl)biphenyl-4-yl]-2-oxo-1-oxa-7-azaspiro[4.5]dec-3-en-7-yl}acetic acid

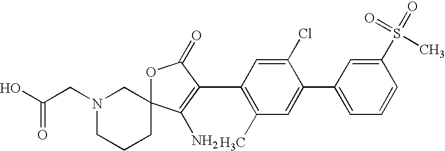

4.78 g of the compound from Example 35 are stirred in 40 ml trifluoroacetic acid for 2.5 h at RT. After evaporation to dryness the residue is treated with potassium carbonate solution (pH 9-10) and extracted three times with ethyl acetate. The organic phases are rejected. Ethyl acetate is then added again to the aqueous phase which is then acidified to pH 1-2 with 1N hydrochloric acid. A precipitate separates which is filtered off by suction, washed with acetonitrile and dried in vacuum at 45° C. (4.1 g crude product). The product is then purified by preparative HPLC in several portions (Method 7). 3.1 g (69% th.) of the product are obtained as a solid.

LC-MS (Method 8): Rₜ=2.11 min, m/z=504 (M+H)⁺

### Exemplary Embodiments

### Example 1

### N-[4′-(4-Amino-2-oxo-1-oxaspiro[4.5]dec-3-en-3-yl)-2′-chloro-5′-methylbiphenyl-3-yl]methane sulphonamide

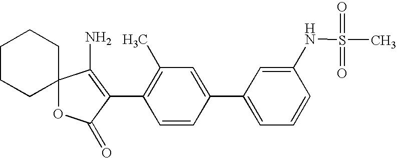

70.0 mg (0.19 mmol) 4-amino-3-(4-bromo-5-chloro-2-methylphenyl)-1-oxaspiro[4.5]dec-3-en-2-one, 60.9 mg (0.28 mmol) {3-[(methylsulphonyl)amino]phenyl}boronic acid and 184.6 mg (0.57 mmol) caesium carbonate are suspended in a mixture of 3 ml dimethoxyethane, 1 ml ethanol and 2 ml water. Argon is then passed through the mixture for 30 min. The suspension is treated with 17.5 mg (0.02 mmol) tetrakis(triphenylphosphine)palladium(0) under argon, the reaction is heated to 50° C. and stirred overnight at this temperature. For work-up the solvent is completely removed with a rotary evaporator and after purification by preparative RP-HPLC (Method 6) 34 mg (38% th.) of the target compound are obtained.

LC-MS (Method 3): Rₜ=2.35 min, m/z=461 (M+H)⁺

¹H NMR (400 MHz, DMSO-d₆), δ=9.89 (s, 1H), 7.42 (t, 1H), 7.28 (s, 2H), 7.22 (m, 2H), 7.16 (d, 1H), 7.05 (bs, 2H), 3.04 (s, 3H), 1.17 (s, 3H), 1.92 (m, 2H), 1.78-1.45 (m, 7H), 1.25 (m, 1H).

### General Method 1: Suzuki Coupling

The aryl halide 4-amino-3-(4-bromo-5-chloro-2-methylphenyl)-1-oxaspiro[4.5]dec-3-en-2-one (0.19 mmol, 1.0 equivalent) is dissolved in 2 ml dimethoxyethane, the solution is purged with argon and then the boronic acid (0.21 mmol, 1.1 equivalents), palladium(II)-acetate (5.66 μmol, 0.03 equivalents), 2-dicyclohexylphosphino-2′,4′,6′-triisopropyl-1,1′-biphenyl (13.22 mmol, 0.07 equivalents) and caesium carbonate (0.57 mmol, 3.0 equivalent) are added. The reaction is stirred in an argon atmosphere for 12 hours at 50° C. For work-up the precipitate is removed by filtration and the target compound is obtained after purification by preparative RP-HPLC (Method 6).

### General Method 2: Suzuki Coupling

The aryl halide (120 mg, 0.22 mmol) is dissolved in 3 ml dimethoxyethane, 2 ml water and 1 ml ethanol, the solution is purged with argon and then 3-methylsulphonylphenylboronic acid (50 mg, 0.25 mmol), tetrakis(triphenylphosphine)palladium (0) (10 mg, 0.01 mmol) and caesium carbonate (218 mg, 0.67 mmol) are added. The reaction is stirred in an argon atmosphere for 12-18 hours at 50° C. If the conversion is incomplete the respective amounts of 3-methylsulphonylphenylboronic acid, tetrakis(triphenylphosphine)palladium (0) and caesium carbonate are again added and stirring is continued for a further 12-18 h at RT.

### Work-up:

A) the precipitate is filtered off by suction and where appropriate purified by preparative HPLC (Method 7) or silica gel chromatography.
B) The reaction is poured onto 5 ml 1N hydrochloric acid and extracted with methylene chloride. The combined organic extracts are dried and evaporated in vacuum. The target compound is obtained after fine purification by preparative RP-HPLC (Method 6).
C) Water is added and extracted with ethyl acetate. The combined organic phases are dried and evaporated in vacuum. The target compound is obtained after fine purification by preparative RP-HPLC (Method 7).
D) After acidification with 5 M acetic acid the reaction solution is purified directly by preparative HPLC (Method 7).

Examples 2 to 14 are prepared by the general working procedure 1 or 2.

| | | Yield | |
|---|---|---|---|
| | | [%] th. | |
| Example | Structure | Method | Analytical data |
| 2 | 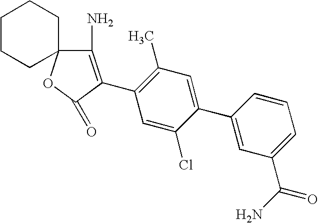 | 43 GWP 1 | LC-MS (Method 2): Rₜ = 2.26 min, m/z = 411 (M + H)⁺. |
| 3 | 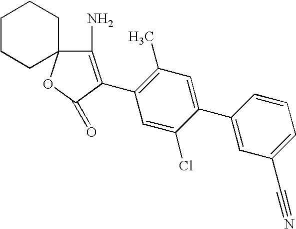 | 35 GWP 1 | LC-MS (Method 2): Rₜ = 2.33 min, m/z = 393 (M + H)⁺. |
| 4 | 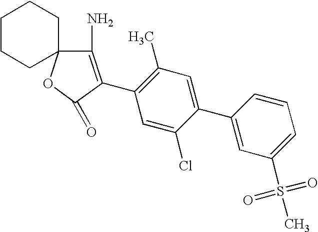 | 39 GWP 1 | LC-MS (Method 2): Rₜ = 2.12 min, m/z = 446 (M + H)⁺: |
| 5 | 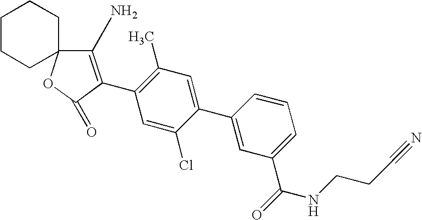 | 39 GWP 1 | LC-MS (Method 3): Rₜ = 2.28 min, m/z = 464 (M + H)⁺. |
| 6 | 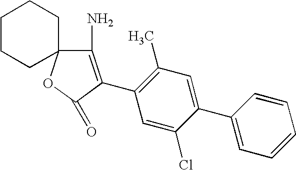 | 71 GWP 1 | LC-MS (Method 2): Rₜ = 2.45 min, m/z = 368 (M + H)⁺. |
| 7 | 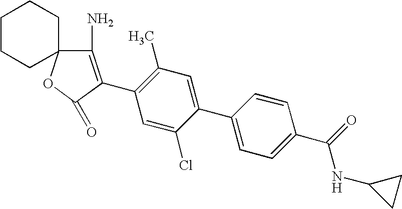 | 56 GWP 1 | LC-MS (Method 3): Rₜ = 2.33 min, m/z = 451 (M + H)⁺. |
| 8 | 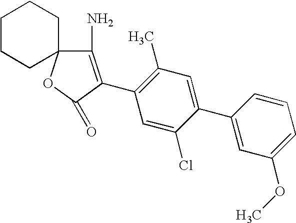 | 31 GWP 1 | LC-MS (Method 2): Rₜ = 2.43 min, m/z = 398 (M + H)⁺. |
| 9 | 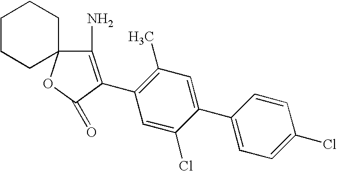 | 43 GWP 1 | LC-MS (Method 2): Rₜ = 2.64 min, m/z = 402 (M + H)⁺. |
| 10 | 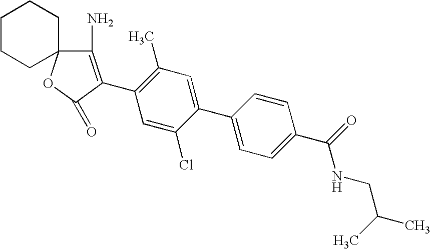 | 14 GWP 1 | LC-MS (Method 3): Rₜ = 2.52 min, m/z = 467 (M + H)⁺. |
| 11 | 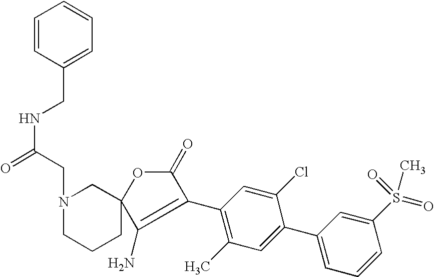 | 60 GWP 2C | LC-MS (Method 2): Rₜ = 1.79 min, m/z = 594 (M + H)⁺. |
| 12 | 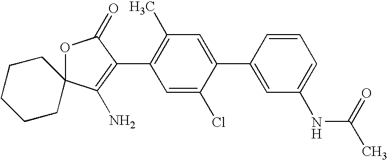 | 46 GWP 1 | LC-MS (Method 3): Rₜ = 2.37 min, m/z = 425 (M + H)⁺; ¹H NMR (400 MHz, DMSO- d₆): δ = 7.55 (s, 1H), 7.51 7.54 (m, 1H), 7.33-7.40 (m, 1H), 7.24 (d, 1H), 7.0-7.1 (m, 3H), 2.17 (s, 3H), 2.05 (s, 3H), 1.86-1.98 (m, 2H), 1.50-1.75 (m, 7H), 1.20-1.33 (m, 1H). |
| 13 | 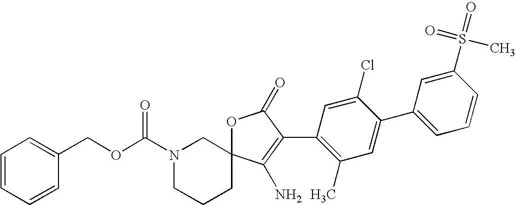 | 76 GWP 2A | LC-MS (Method 8): Rₜ = 2.53 min, m/z = 581 (M + H)⁺; |
| 14 | 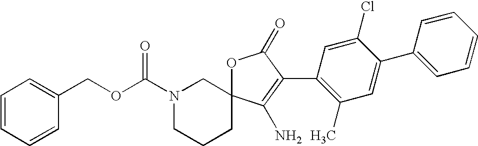 | 75 (GWP 2D) | LC-MS (Method 9): Rₜ = 2.82 min, m/z = 503 (M + H)⁺; |

### Example 15

### 4-Amino-3-(3′-amino-2-chloro-5-methylbiphenyl-4-yl)-1-oxaspiro[4.5]dec-3-en-2-one

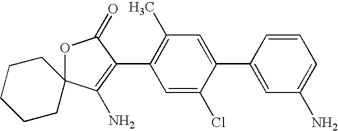

4.714 g (11.1 mmol) of the compound from example 12 are stirred in 50 ml 5N hydrochloric acid and 5 ml Dioxan for 1 h at 60° C. The suspension is then treated with 30 ml acetonitrile and heated under reflux for 2 h. After evaporation to dryness the residue (6.6 g) is partitioned between saturated sodium hydrogen carbonate solution and ethyl acetate and extracted twice more with ethyl acetate. The combined organic phases are dried, evaporated and chromatographed on silica gel (eluent dichloromethane/methanol (100:0) to (40:1)). 3.87 g (89% th.) of the product are obtained.

LC-MS (Method 1): Rₜ=2.08 min, m/z=383 (M+H)⁺

¹H NMR (400 MHz, DMSO-d₆), δ=7.17-7.22 (m, 2H), 7.00-7-11 (m, 3H), 6.50-6-61 (m, 3H), 5.2 (b, 2H), 2.15 (s, 3H) 1.37-1.49 (m, 2H), 1.50-1.75 (m, 7H), 1.20-1-33 (m, 1H).

### Example 16

### N-[4′-(4-Amino-2-oxo-1-oxaspiro[4.5]dec-3-en-3-yl)-2′-chloro-5′-methylbiphenyl-3-yl]ethane sulphonamide

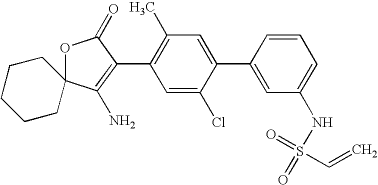

785 mg (2 mmol) of the compound from Example 15 are dissolved in 25 ml DME and stirred for 1 h at 55° C. with 25 mg (0.2 mmol) DMAP, 0.7 ml (4 mmol) DIPEA and 0.325 ml (3 mmol) 2-chloroethylsulphonyl chloride. After cooling 8 ml 1N hydrochloride are added to the mixture which is then evaporated to a few millilitres. More water is added and after a few minutes treatment in an ultrasonic bath the precipitate is filtered off. The residue (1 g) is flash chromatographed with dichloromethane/ethyl acetate (2:1). 700 mg (67% th.) of the product are obtained as a solid.

LC-MS (Method 1): Rₜ=2.39 min

MS (ESIpos): m/z=473 (M+H)⁺

¹H NMR (400 MHz, DMSO-d₆): δ=10.15 (s, 1H), 7.40 (t, 1H), 7.22-7.27 (m, 3H), 7.19 (d, 1H), 7.12 (d, 1H) 7.03 (m, 2H) 6.82 (dd, 1H), 6.05-6.17 (dd, 1H), 2.17 (s, 3H), 1.88-2.00 (m, 2H), 1.51-1.76 (m, 7H), 1.22-1.33 (m, 1H).

### Example 17

### 2-({[4′-(4-Amino-2-oxo-1-oxaspiro[4.5]dec-3-en-3-yl)-2′-chloro-5′-methylbiphenyl-3-yl]amino}-sulphonyl)-N,N-dimethylethanammonium formate

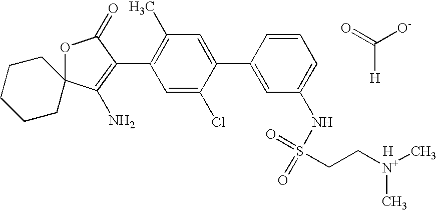

40 mg (0.085 mmol) of the compound from Example 16 are dissolved in 1 ml ethanol, treated with 0.48 ml (3.8 mmol) of a 40% aqueous dimethylamine solution and stirred for 1.5 h at RT. After evaporating to dryness and purification by preparative HPLC (Method 7) 24.5 mg (51% th.) of product are obtained.

LC-MS (Method 9): Rₜ=1.71 min

MS (ESIpos): m/z=518 (M+H)⁺

¹H NMR (400 MHz, DMSO-d₆): δ=9.9 (b, 1H), 8.13 (s, 1H), 7.92 (t, 1H), 7.21-7.31 (m, 4H), 7.13 (d, 1H), 7.03 (b, 2H), 3.27 (m, superimposed by water signal), 2.62-2.68 (m, 2H), 2.18 (s, 3H), 2.09 s, 6H), 1.88-1.98 (m, 2H), 1.52-1.78. (m, 7H), 1.20-1.37 (m, 1H).

The following compounds are prepared by analogy to the method for Example 17 (the reaction time for the preparation of the compound from Example 18 is 1d):

| | | Reagent | |
|---|---|---|---|
| | | Yield | |
| Example | Structure | [%] th. | Analytical data |
| 18 | 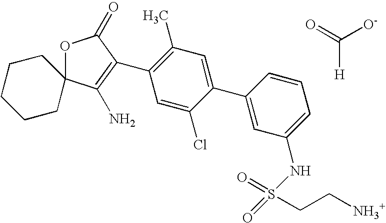 | 7 M methanol. ammonia (400 eq.), 31 | LC-MS (Method 9): Rₜ = 1.68 min, m/z = 490 (M + H)⁺. |
| 19 | 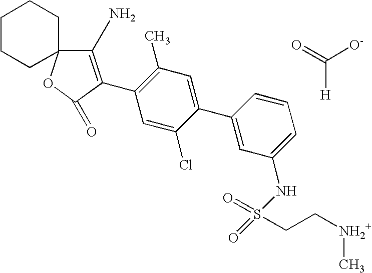 | methylamine, 40% in water (50 eq.), 13 | LC-MS (Method 9): Rₜ = 1.68 min, m/z = 504 (M + H)⁺. |
| 20 | 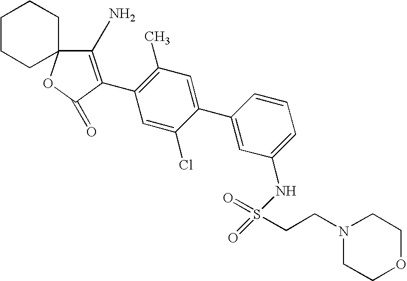 | morpholine (4 eq.) 39 | LC-MS (Method 9): Rₜ = 1.90 min, m/z = 560 (M + H)⁺. |
| 21 | 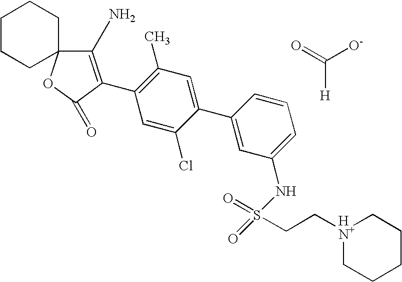 | piperidine (10 eq.) 58 | LC-MS (Method 1): Rₜ = 1.73 min, m/z = 558 (M + H)⁺. |
| 22 | 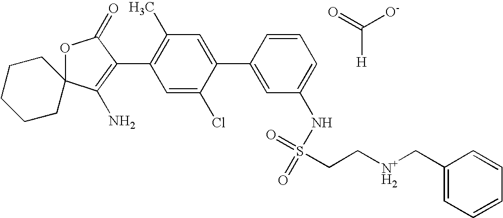 | benzylamine (10 eq.) 37 | LC-MS (Method 1): Rₜ = 1.84 min, m/z = 580 (M + H)⁺. |
| 23 | 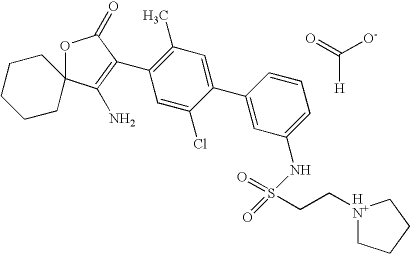 | pyrrolidine (20 eq.) | LC-MS (Method 1): Rₜ = 1.74 min, m/z = 544 (M + H)⁺. |
| 24 | 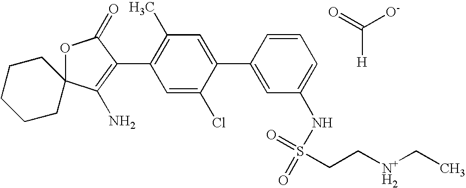 | 2 M methanol. ethylamine (20 eq.) | LC-MS (Method 1): Rₜ = 1.68 min, m/z = 518 (M + H)⁺. |

### Example 25

### 4-[3-({[4′-(4-Amino-2-oxo-1-oxaspiro[4.5]dec-3-en-3-yl)-2′-chloro-5′-methylbiphenyl-3-yl]amino}-sulphonyl)propyl] morpholin-4-ium formate

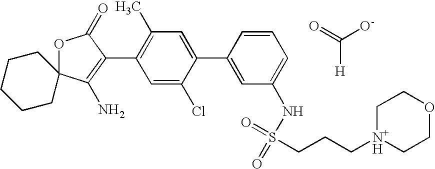

30 mg (0.053 mmol) of the compound from Example 11A are dissolved in 1 ml THF, treated with 18 μl (0.21 mmol) morpholine and stirred for 18 h at RT. A further 18 μl morpholine are then added and the mixture is stirred for 4 h at 60° C. The mixture is evaporated and purified by preparative HPLC (Method 7). 17.5 mg (53% th.) of the product are obtained.

LC-MS (Method 1): Rₜ=1.55 min, m/z=574 (M+H)⁺

¹H NMR (400 MHz, DMSO-d₆+CD₃OD): δ=8.16 (s, 1H), 7.42 (t, 1H), 7.30 (m, 1H), 7.2-7.26 (m 3H), 7.13 (d, 1H), 7.03 (b, 2H), 3.22 (m, superimposed by water), 2.32 (t, 2H), 2.15-2.24 (m, 4H), 1.54-1.98 (m, 9H), 1.20-1.34 (m, 1H).

The following compounds are prepared from the compound from Example 10A and the respective boronic acid by analogy to GWP 2D. Deviation from GWP 2D: solvent dioxan/water, base: sodium carbonate, temp.: 80° C., purification: HPLC (Method 13) direct from the reaction solution after filtration.

| Example | Structure | Analytical data |
|---|---|---|
| 26 | 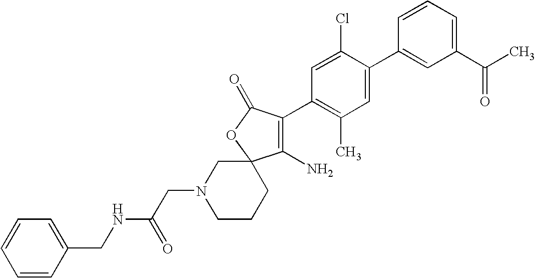 | LC-MS (Method 12): Rₜ = 1.75 min, m/z = 558 (M + H)⁺. |
| 27 | 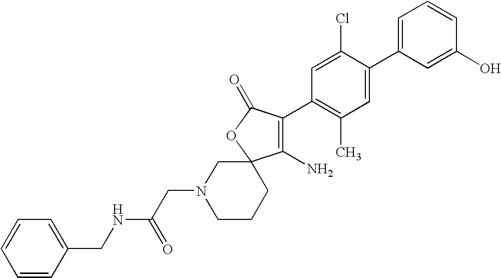 | LC-MS (Method 12): Rₜ = 1.63 min, m/z = 532 (M + H)⁺. |
| 28 | 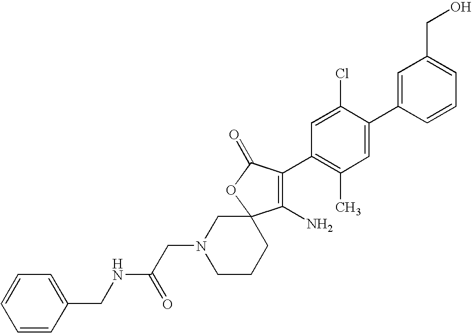 | LC-MS (Method 12): Rₜ = 1.63 min, m/z = 545 (M + H)⁺. |
| 29 | 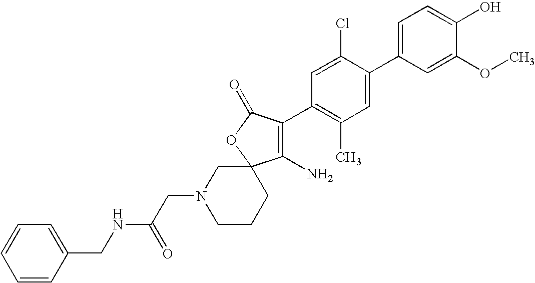 | LC-MS (Method 12): Rₜ = 1.65 min, m/z = 562 (M + H)⁺. |
| 30 | 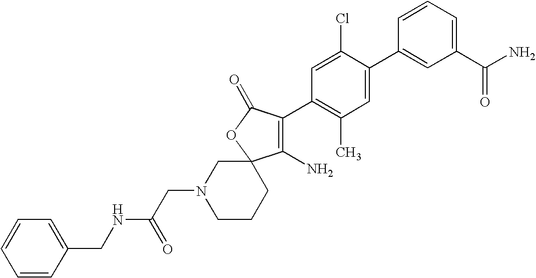 | LC-MS (Method 12): Rₜ = 1.98 min, m/z = 559 (M + H)⁺. |
| 31 | 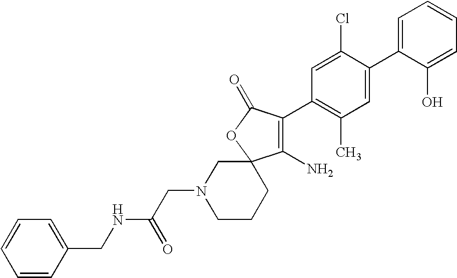 | LC-MS (Method 12): Rₜ = 1.68 min, m/z = 532 (M + H)⁺. |
| 32 | 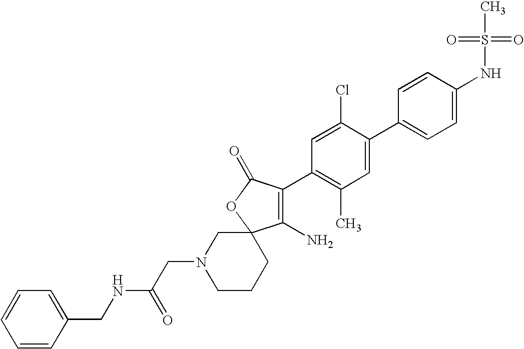 | LC-MS (Method 12): Rₜ = 1.62 min, m/z = 609 (M + H)⁺. |
| 33 | 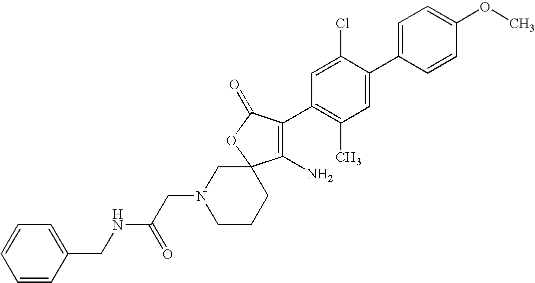 | LC-MS (Method 12): Rₜ = 1.63 min, m/z = 546 (M + H)⁺. |
| 34 | 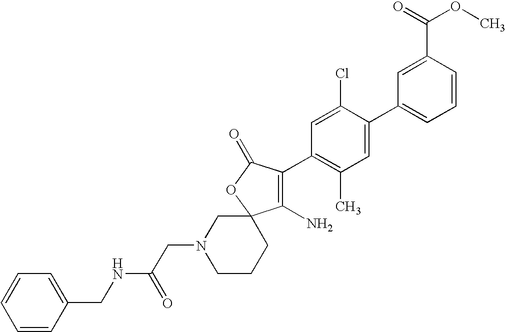 | LC-MS (Method 12): Rₜ = 1.81 min, m/z = 574 (M + H)⁺. |

### Example 35

### Tert.-Butyl-{4-amino-3-[2-chloro-5-methyl-3′-(methylsulphonyl)biphenyl-4-yl]-2-oxo-1-oxa-7-azaspiro[4.5]dec-3-en-7-yl}acetate

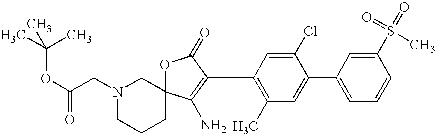

4.0 g (9 mmol) of the compound from Example 12A are dissolved in DMF, treated with 2.0 g (9.9 mmol) tert.-butyl bromoacetate and 2.74 g (19.8 mmol) potassium carbonate and stirred for 1 h at 50° C. The reaction mixture is poured with stiffing into 500 ml water, the precipitate is filtered off, washed with water and dried in vacuum at 45° C. 4.39 g (85% th.) of the product is obtained as a solid.

LC-MS (Method 9): Rₜ=1.85 min, m/z=561 (M+H)⁺

The compounds of Examples 36 to 47 of the table are prepared in an analogous manner by the following general working procedure: 0.1 mmol of the compound from Example 12A dissolved in DMF and 0.1 mmol of the respective reagents dissolved in DMSO are shaken with 0.2 mmol potassium carbonate for 1d at RT, filtered and purified by preparative HPLC.

| Example | Structure | Analytical data |
|---|---|---|
| 36 | 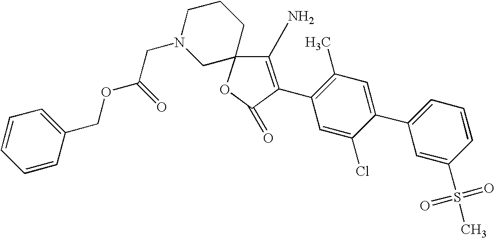 | LC-MS (Method 11): Rₜ = 1.77 min, m/z = 595 (M + H)⁺. |
| 37 | 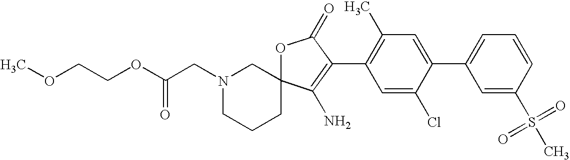 | LC-MS (Method 11): Rₜ = 1.49 min, m/z = 563 (M + H)⁺. |
| 38 | 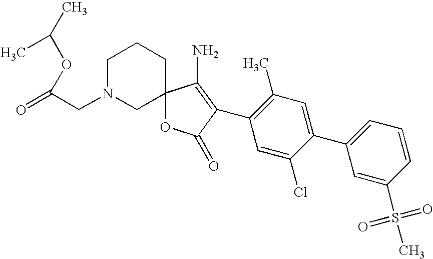 | LC-MS (Method 11): Rₜ = 1.59 min, m/z = 547 (M + H)⁺. |
| 39 | 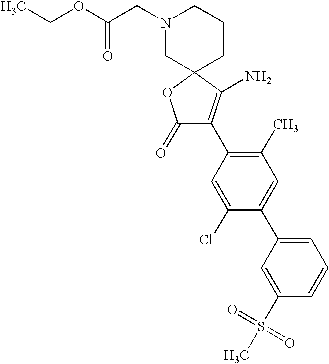 | LC-MS (Method 11): Rₜ = 1.53 min, m/z = 533 (M + H)⁺. |
| 40 | 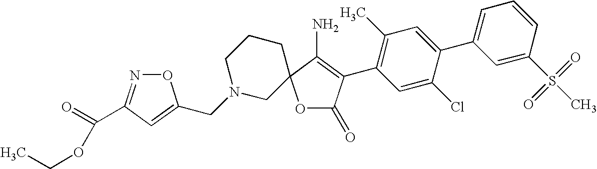 | LC-MS (Method 11): Rₜ = 1.79 min, m/z = 600 (M + H)⁺. |
| 41 | 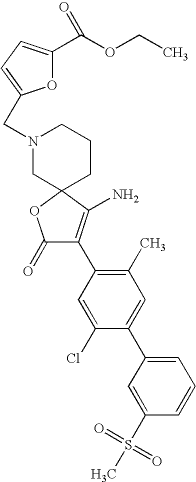 | LC-MS (Method 11): Rₜ = 1.56 min, m/z = 599 (M + H)⁺. |
| 42 | 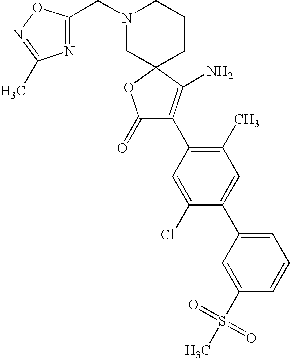 | LC-MS (Method 11): Rₜ = 1.81 min, m/z = 543 (M + H)⁺. |
| 43 | 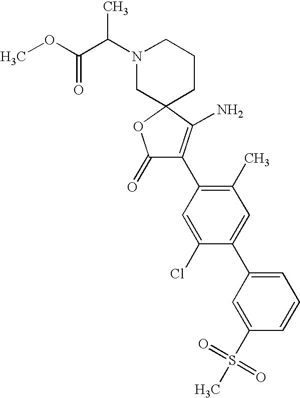 | LC-MS (Method 11): Rₜ = 1.55 min, m/z = 533 (M + H)⁺. |
| 44 | 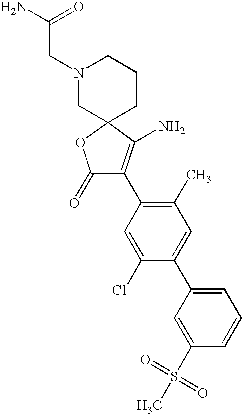 | LC-MS (Method 11): Rₜ = 1.34 min, m/z = 504 (M + H)⁺. |
| 45 | 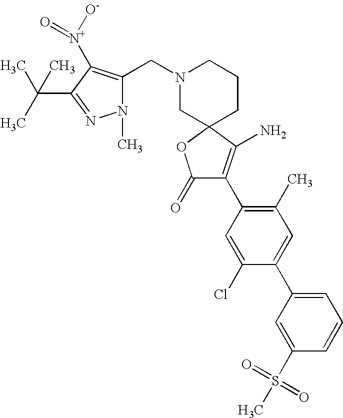 | LC-MS (Method 11): Rₜ = 2.21 min, m/z = 642 (M + H)⁺. |
| 46 | 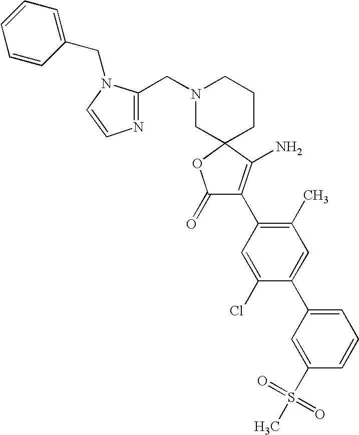 | LC-MS (Method 11): Rₜ = 1.57 min, m/z = 617 (M + H)⁺. |
| 47 | 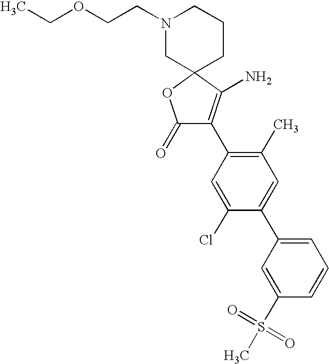 | LC-MS (Method 11): Rₜ = 1.43 min, m/z = 519 (M + H)⁺. |

### Example 48

### 2-{4-Amino-3-[2-chloro-5-methyl-3′-(methylsulphonyl)biphenyl-4-yl]-2-oxo-1-oxa-7-zaspiro[4.5] dec-en-7-yl}-(cyclohexylmethyl)acetamide

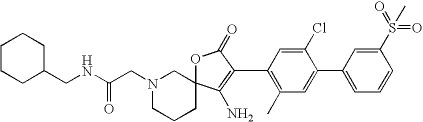

50 mg (0.079 mmol) of the compound from Example 13A are stirred in 2 ml DMSO with 9 mg (0.079 mmol) cyclohexylmethylamine, 20.5 mg (0.159 mmol) DIPEA and 33 mg (0.103 mmol) TBTU for 18 h at RT. After acidification to pH 4-5 and purification by preparative HPLC (Method 7) 12 mg (25% th.) of the product is obtained as a solid.

By analogy to the method for Example 48 the following compounds are prepared from the compound from Example 13A and the respective amines:

| | | LC-MS (Method |
|---|---|---|
| | | 11) |
| | | Rₜ [min] |
| Example | Structure | m/z (M + H)⁺ |
| 49 | 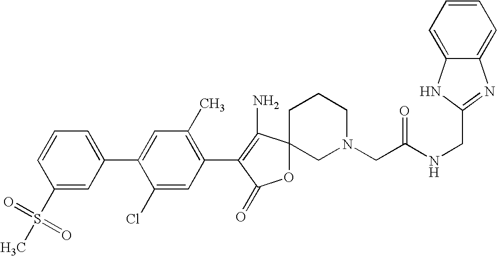 | Rₜ: 1.36 m/z: 634 |
| 50 | 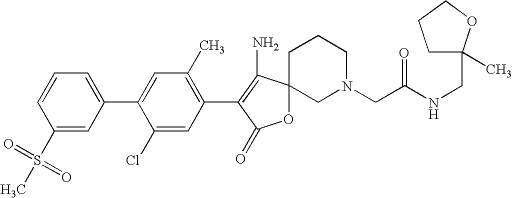 | Rₜ: 1.49 m/z: 602 |
| 51 | 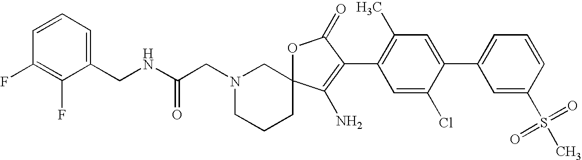 | Rₜ: 1.63 m/z: 630 |
| 52 | 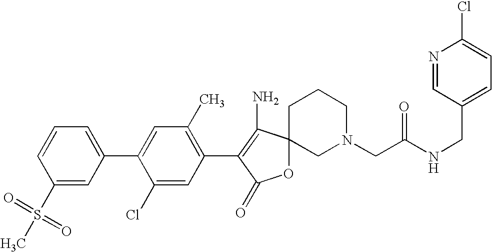 | Rₜ: 1.54 m/z: 629 |
| 53 | 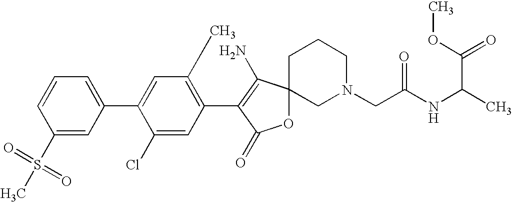 | Rₜ: 1.49 m/z: 590 |
| 54 | 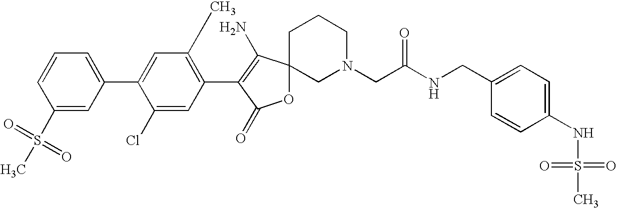 | Rₜ: 1.50 m/z: 687 |
| 55 | 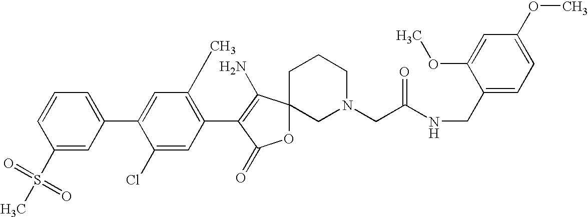 | Rₜ: 1.64 m/z: 654 |
| 56 | 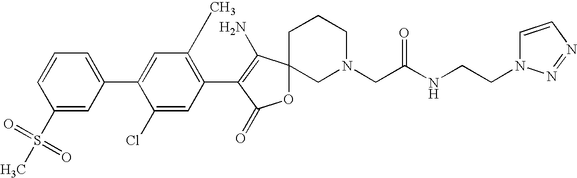 | Rₜ: 1.40 m/z: 599 |
| 57 | 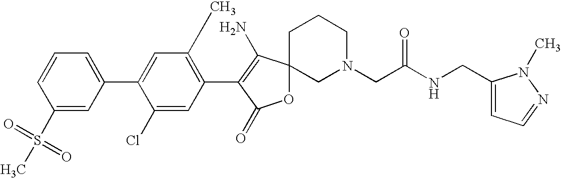 | Rₜ: 1.45 m/z: 598 |
| 58 | 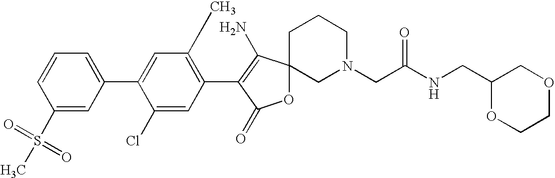 | Rₜ: 1.44 m/z: 604 |
| 59 | 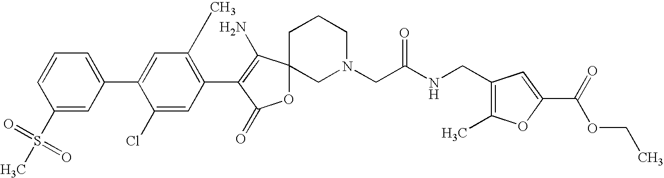 | Rₜ: 1.62 m/z: 670 |
| 60 | 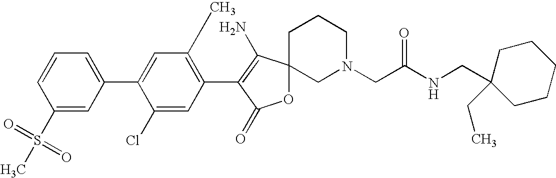 | Rₜ: 1.82 m/z: 628 |
| 61 | 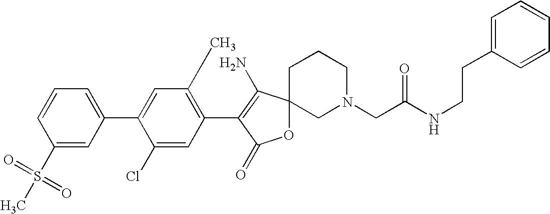 | Rₜ: 1.62 m/z: 608 |
| 62 | 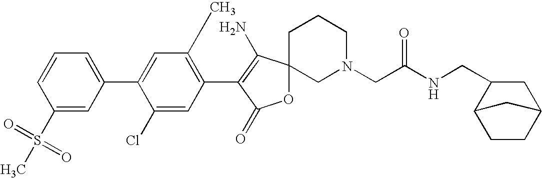 | Rₜ: 1.72 m/z: 612 |
| 63 | 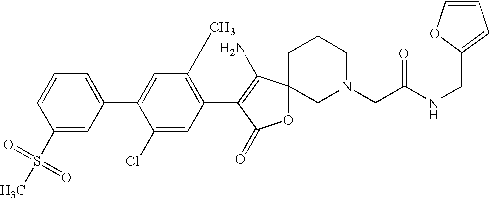 | Rₜ: 1.53 m/z: 584 |
| 64 | 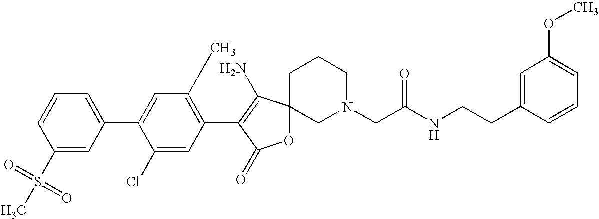 | Rₜ: 1.62 m/z: 638 |
| 65 | 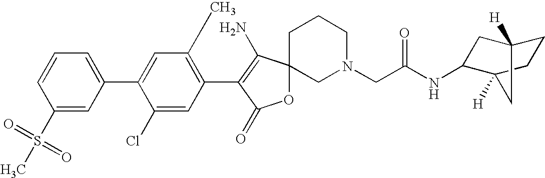 | Rₜ: 1.63 m/z: 598 |
| 66 | 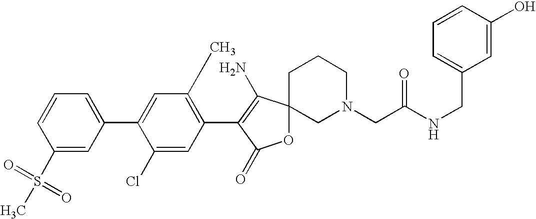 | Rₜ: 1.51 m/z: 610 |
| 67 | 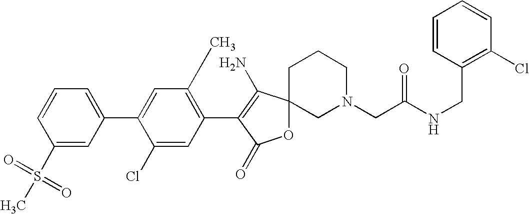 | Rₜ: 1.67 m/z: 628 |
| 68 | 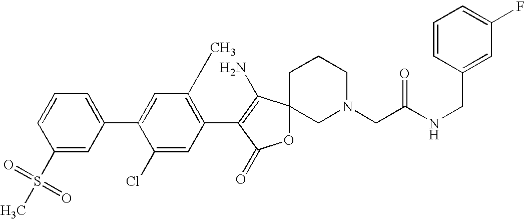 | Rₜ: 1.53 m/z: 612 |
| 69 | 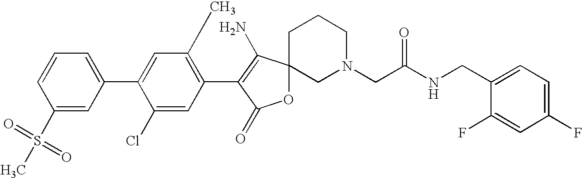 | Rₜ: 1.65 m/z: 630 |
| 70 | 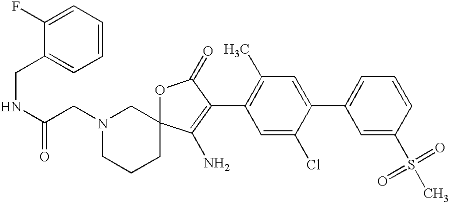 | Rₜ: 1.61 m/z: 612 |
| 71 | 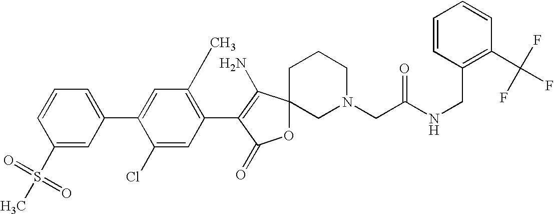 | Rₜ: 1.71 m/z: 662 |
| 72 | 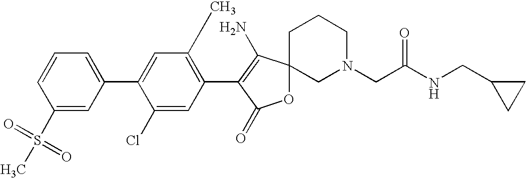 | Rₜ: 1.51 m/z: 558 |
| 73 | 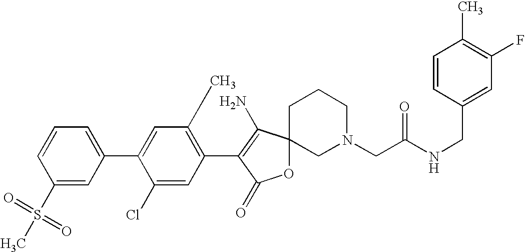 | Rₜ: 1.69 m/z: 626 |
| 74 | 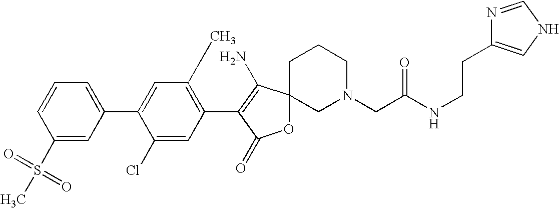 | Rₜ: 1.25 m/z: 598 |
| 75 | 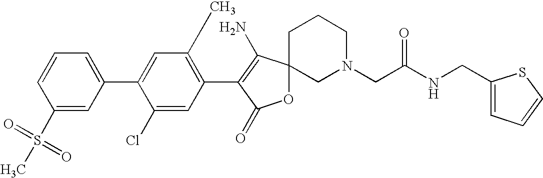 | Rₜ: 1.58 m/z: 600 |
| 76 | 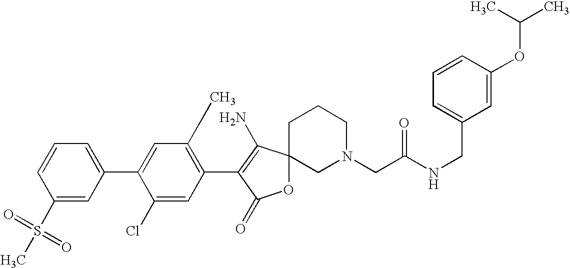 | Rₜ: 1.70 m/z: 652 |
| 77 | 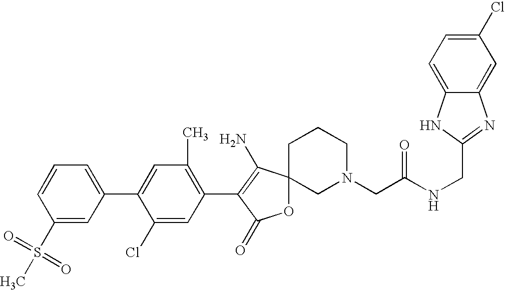 | Rₜ: 1.51 m/z: 668 |
| 78 | 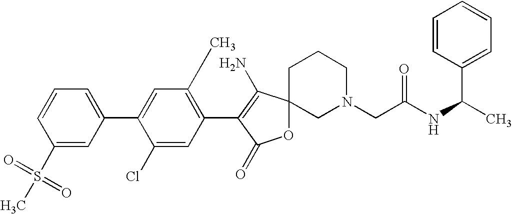 | Rₜ: 1.64 m/z: 608 |
| 79 | 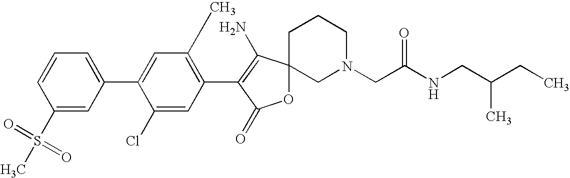 | Rₜ: 1.60 m/z: 574 |
| 80 | 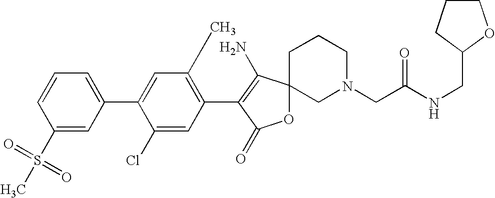 | Rₜ: 1.47 m/z: 588 |
| 81 | 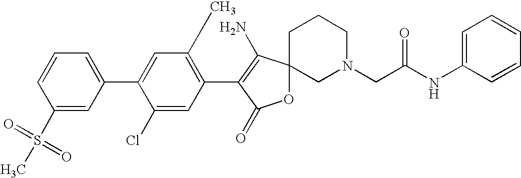 | Rₜ: 1.64 m/z: 580 |

The following compounds are separated into the enantiomers by HPLC on a chiral phase (Method 10). In each case the later eluted enantiomer (Enantiomer 2) is described The absolute configuration is assigned by analogy.

| | | Rₜ | |
|---|---|---|---|
| | | (Meth. | |
| | | 10) [min] | |
| | | Enantio. | |
| Example | Structure | 2 | Analytical data |
| 82 | 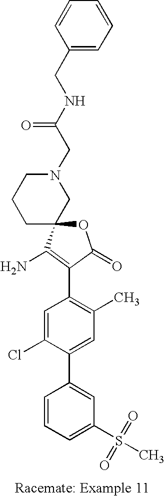 | 12.98 | LC-MS (Method 8): Rₜ = 2.73 min, m/z = 594 (M + H)⁺; ¹H NMR (500 MHz, DMSO- d₆): 8.3-8.2 (b, 1H), 8.0-7.93 (m, 2H), 7.82 (d, 1H), 7.8-7.74 (m, 1H), 7.4 (s, 1H), 7.37-7.18 (m, 6H), 7.12 (b, 2H), 4.4-4.25 (m, 2H), 3.3 (s, 3H), 3.18-3.04 (m, 2H), 2.83-2.73 (m, 2H), 2.70-2.62 (m, 1H), 2.4-2.3 (m, 1H), 2.2 (s, 3H), 2.0-1.84 (m, 2H), 1.72-1-61 (m, 2H) |
| 83 | 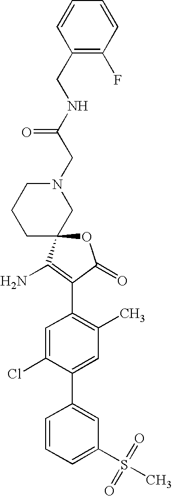 | 15.54 | LC-MS (Method 8): Rₜ = 2.82 min, m/z = 612 (M + H)⁺; ¹H NMR (400 MHz, DMSO- d₆): 8.28-8.18 (b, 1H), 8.02- 7.93 (m, 2H), 7.84 (d, 1H), 7.8- 7.74 (m, 1H), 7.4 (s, 1H), 7.37- 7.25 (m, 3H), 7.18-7.05 (m, 4H), 4.45-4.28 (m, 2H), 3.3 (s, 3H), 3.18-3.07 (m, 2H), 2.85-2.75 (m, 2H), 2.74-2.66 (m, 1H), 2.4-2.3 (m, 1H), 2.19 (s, 3H), 2.04-1.83 (m, 2H), 1.74-1-61 (m, 2H) |
| 84 | 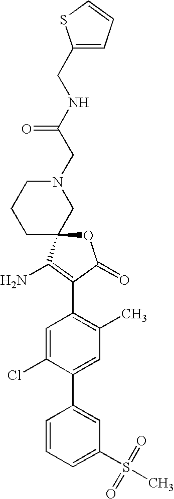 | 13.00 | LC-MS (Method 9): Rₜ = 1.88 min, m/z = 600 (M + H)⁺; ¹H NMR (400 MHz, DMSO- d₆): 8.34-8.24 (b, 1H), 8.01- 7.93 (m, 2H), 7.86-7.74 (m, 2H), 7.4 (s, 1H), 7.35 (m, 1H), 7.30 (m, 1H), 7.15-7.05 (b, 2H), 7..0-6.9 (m, 1H) 4.57-4.42 (m, 2H), 3.3 (s, 3H), 3.15-3.03 (m, 2H), 2.84-2.66 (m, 3H), 2.4-2.27 (m, 1H), 2.19 (s, 3H), 2.05-1.82 (m, 2H), 1.75-1-61 (m, 2H) |
| 85 | 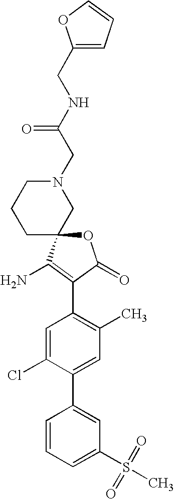 | 12.17 | LC-MS (Method 8): Rₜ = 2.55 min, m/z = 584 (M + H)⁺; ¹H NMR (400 MHz, DMSO- d₆): 8.15-8.08 (b, 1H), 8.0-7.94 (m, 2H), 7.86-7.75 (m, 2H), 7.55 (s, 1H), 7.4 (s, 1H), 7.3 (s, 1H), 7.16-7.07 (b, 2H), 6.38 (m, 1H), 6.22 (m 1H), 4.39- 4.26 (m, 2H), 3.3 (s, 3H), 3.15- 3.03 (m, 2H), 2.84-2.66 (m, 3H), 2.4-2.28 (m, 1H), 2.2 (s, 3H), 2.03-1.82 (m, 2H), 1.75- 1-62 (m, 2H) |

### B) EVALUATION OF PHYSIOLOGICAL ACTIVITY

The suitability of the compounds of the invention for the treatment of diseases caused by retroviruses can be shown by the following assay systems:

In vitro Assays

### Biochemical Protease Assay

For the determination of their in vitro activity on HIV protease the test substances are dissolved in DMSO and serially diluted. In each case 0.5 μl of substance dilution, 20 μl of 0.2−1 nM HIV-1 protease wild type or mutant protein (e.g. multiresistant isolate “35513”: L10I, I15V, L19I, K20R, E35D, M36I, R41K, I54V, L63P, H69K, A71V, T74P, I84V, L89M, L90M, 193L, AscoProt Biotech, Prague, Czech Republic) in buffer 1 (50 mM sodium acetate pH 4.9, 0.02% BSA, 0.1 mM EDTA, 0.5 mM DTT) and 20 μl of 8 μM substrate (M1865 from Bachem, Bubendorf, Switzerland; Matayoshi et al., Science 1990, 247, 954-8) in buffer 1 are added successively to a 384 well microtiter plate (Greiner, Frickenhausen, Germany), incubated for 60-180 minutes at 32° C. and the fluorescence is measured (e.g. Tecan Safire, 340 nm extinction, 520 nm emission). IC₅₀ values are determined by graphical plotting of the substance concentration against the percentage inhibition. Table 1 shows IC₅₀ values for HIV-1 protease wild type protein.

TABLE 1

| Example | IC₅₀ |
|---|---|
| number | [nM] |
| 20 | 510 |
| 27 | 14 |
| 51 | 2.6 |
| 79 | 85 |
| 82 | 11 |
| 84 | 2.5 |

### Assembly Assay

The assembly assay records the late phase of HIV replication.

Day 1: 4×10e7 HEK293T cells of a logarithmically growing culture are seeded in 40 ml of medium (D-MEM with 4500 mg/l glucose, 10% inactivated FKS, 2 mM glutamine, 100 μg/ml penicillin/streptamycin) in a 225 cm² culture flask and incubated overnight in a cell culture incubator.

Day 2: The cells are co-transfected with each time 40 μg of pGJ3-RT K103N/Y181C and pcz-VSV-Gwt (provided by Jassoy) (according to Lipofectamine 2000 Protocol from Invitrogen). The transfection assay is incubated for 5 h in a cell incubator. The cells are then trypsinated and counted. The transfected cells are adjusted with fresh medium to 3×10e5 cells/ml and 40 μl of the cell suspension per well is seeded onto a white 384 MTP (Greiner) which is already charged with 10 μl/well test of a substance solution (test substances in medium without pen/strep). HEK293T cells of a logarithmically growing culture are adjusted to a concentration of 3.5×10e5 cells/ml with medium and 40 μl per well of this cell suspension are distributed onto a white 384 MTP and incubated overnight in a cell culture incubator.

Day 3: 24 h after seeding the transfected cells onto the substance plate 10 μl of supernatant are taken from each well with which the cells seeded the previous day are infected. The infected cells are incubated overnight in the cell incubator. The luciferase activity of the transfected cells on the substance plate is measured in a luminometer after the addition of 20 μl of luciferase/triton buffer.

Day 4: The luciferase activity of the infected cells is measured in a luminometer after the addition of 20 μl of luciferase/triton buffer.

The CC₅₀ value of a test substance is derived from the luciferase activity of the treated transfected cells in comparison to the untreated control cells.

The EC₅₀ value of a test substance is derived from the luciferase activity of the infected cells in comparison to the infected control cells.

### HIV Infection in Cell Culture

The HIV test is carried out with modifications according to the method of Pauswels et al. [cf. Journal of Virological Methods 1988, 20, 309-321].

Primary human blood lymphocytes (PBL's) are enriched via Ficoll-Hypaque and stimulated in RPMI 1640 medium (from Gibco, Invitrogen Corporation, Karlsruhe, Germany), 20% foetal calf serum with phythaemagglutinin (90 μg/ml) and interleukin-2 (40 U/ml). For the infection with infectious HIV the PBL's are pelleted and the cell pellet is subsequently suspended in 1 ml of a suitably diluted HIV virus adsorption solution and incubated for one hour at 37° C. (pellet infection). Non-adsorbed virus is subsequently removed by centrifugation and the infected cells are transferred into test plates (e.g. 96 well microtiter plates) which contain the test substances in a suitable dilution.

Alternatively e.g. HIV susceptible, permanent H9 cells (ATCC or NAIAD, USA) are used in place of normal human blood lymphocytes to test the antiviral effects of the compounds of the invention. Infected H9 cells are cultured in RPMI 1640 medium, 2% and/or 20% fetal calf serum for test purposes.

The virus adsorption solution is centrifuged and the infected cell pellet taken up in growth medium so that it is adjusted to 1×10⁵ cells per ml. The cells infected in this way are pipetted into the wells of 96 well microtiter plates at about 1×10⁴ cells/well (pellet infection). Alternatively the HIV is pipetted in separately after the preparation of the substance dilution in the microtiter plates and after the addition of the cells (supernatant infection).

The first vertical row of the microtiter plate contains only growth medium and cells that are not infected but are otherwise treated exactly as described above (cell control). The second vertical row of the microtiter plate contains only HIV infected cells in growth medium (virus control). The remaining wells contain the compounds of the invention in different concentrations, starting from the 3rd vertical row of the microtiter plate from which on the test substances are diluted 2¹⁰ times in double steps.

Alternatively supernatant infections are carried out (see above) in which the cells are seeded into 96 well plates. The HIV virus is then added in a volume of 50 μl.

The test assays are incubated at 37° C. until the formation of syncitia typical for HIV appears in the untreated virus control (between day 3 and 6 after infection), which are then evaluated either microscopically or by the p24 ELISA detection method (Vironostika, BioMerieux, The Netherlands) or photometrically or fluorometrically by Alamar Blue indicator dye. Under these test conditions about 20-100 syncitia result in the untreated virus control, whereas no syncitia appear in the untreated cell control. Correspondingly the ELISA Test shows values smaller than 0.1 for the cell controls and values between 0.1 and 2.9 for the virus controls. The photometric evaluation of the Alamar Blue treated cells shows extinctions smaller than 0.1 for the cells controls, whereas the virus controls have values between 0.1 and 3 at corresponding wave lengths.

The IC₅₀ values are determined as the concentration of the treated and infected cells at which 50% (about 20-100 syncitia) of the virus-induced syncitia are suppressed by the treatment with the compounds of the invention. The cut-off values are correspondingly set in the ELISA test and in the photometric or fluorometric determination with Alamar Blue. In addition to the determination of the antiviral effects the treated cell cultures are also investigated microscopically with respect to cytotoxic, cytostatic or cytological changes as well as with respect to solubility. Active compounds that show cell-changing, cytotoxic effects in the concentration range of activity are not assessed for their antiviral activity.

It is found that the compounds of the invention protect HIV-infected cells from virus-induced cell destruction.

In vivo Assay

The antiviral activity of a substance, that is the ability to reduce the titer of human immunodeficiency virus (HIV), is tested in the murine HIV model.

Human cells are infected with HIV in vitro. After the incubation the infected cells are transferred onto a collagen sponge (Gelfoam®) and transplanted subcutaneously onto the backs of immunodeficient mice. At least three groups each of 5-10 animals are used in the in vivo assay. One group represents the negative control group (placebo). One group is treated with a known antivirally active substance (e.g. Sustiva) and serves as positive control group. In further groups the substance with unknown activity is tested. For each additional test assay a group each of 5-10 animals are included. The animals are treated in different ways (e.g. orally twice daily) for a few days (e.g. 4 days). The animals are subsequently sacrificed. Blood and tissue samples can be taken for further analysis (e.g. pharmacokinetics). The collagen sponge is removed and enzymatically digested so that the cells remain. The RNA and DNA is isolated from these cells and the viral load determined, for example, by quantitative PCR.

The antiviral activity of a substance is determined relative to the activity in the placebo and positive controls with the assistance of statistical methods.

### C) EXEMPLARY EMBODIMENTS FOR PHARMACEUTICAL COMPOSITIONS

The compounds of the invention can be converted into pharmaceutical preparations as follows:

### Tablets:

### Composition:

100 mg of the compound of example 1, 50 mg of lactose (monohydrate), 50 mg of corn starch (native), 10 mg of polyvinylpyrolidone (PVP 25) (BASF, Ludwigshafen, Germany) and 2 mg of magnesium stearate.

Tablet weight 212 mg. diameter 8 mm, radius of curvature 12 mm.

### Preparation:

The mixture of the compound of the invention, lactose and starch is granulated with a 5% solution (m/m) of the PVP in water. After drying the granules are mixed with the magnesium stearate for 5 minutes. This mixture is compressed in a conventional tablet press (tablet format see above) A pressure of 15 kN is used as guideline for the compression.

Solution which can be Administered Orally:

### Composition

500 mg of the compound from example 1, 2.5 g of polysorbate and 97 g of polyethyleneglycol 400. A single dose of 100 mg of the compound of the invention corresponds to 20 g of oral solution.

### Preparation

The compound of the invention is suspended in the mixture of polyethyleneglycol and polysorbate with stiffing. The stiffing procedure is continued until the dissolution of the compound of the invention is complete.

i.v. Solution:

The compound of the invention is dissolved in a concentration below saturation solubility in a physiologically acceptable solvent (e.g. isoton. saline, glucose solution 5%, PEG 400 solution 30%). The solution is sterilised by filtration and dispersed into sterile and pyrogen-free injection containers.

1. Compounds of the formula 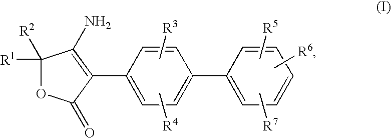
in which
R¹ and R² together with the carbon atom to which they are bonded form a group of formula 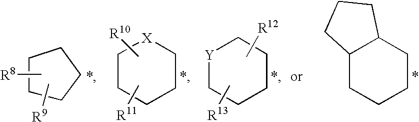
whereby
* represents the carbon atom to which R¹ and R² are bonded, represents the number 1, 2 or 3,
X for an oxygen atom, a sulphur atom or NR¹⁴,
whereby
R¹⁴ represents C₁-C₆ alkyl, C₂-C₄ alkenyl, C₁-C₄ alkylsulphonyl, benzylsulphonyl, —(CH₂)ₒCOR¹⁶, —(CH₂)ₚCONR¹⁷R¹⁸, —(CH₂)_{q}NR²⁴COR²⁵ or —(CH₂)ᵥNR²⁶SO₂R²⁷,
whereby alkyl, alkenyl and alkylsulphonyl can be substituted with 1 to 2 substituents, whereby the substituents are selected independently of one another from the group consisting of halogen, cyano, hydroxy, trifluoromethyl, hydroxycarbonyl, aminosulphonyl, C₁-C₄ alkoxy, C₁-C₄ alkoxycarbonyl, C₁-C₆ alkylaminocarbonyl, C₁-C₆ alkylaminosulphonyl, benzylaminosulphonyl, C₃-C₇ cycloalkyl, phenyl, 5- to 10-membered heterocyclyl, 5- to 10-membered heteroaryl and —OR²²,
wherein phenyl, heterocyclyl and heteroaryl can be substituted with 1 to 3 substituents, whereby the substituents are selected independently of one another from the group consisting of halogen, cyano, nitro, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl, C₁-C₄ alkoxycarbonyl and benzyl,
and
wherein alkoxy can be substituted with a substituent selected from the group consisting of halogen, cyano, trifluoromethyl, hydroxy, hydroxycarbonyl, aminocarbonyl, aminosulphonyl, C₁-C₄ alkoxy, C₁-C₄ alkoxycarbonyl, C₁-C₄ alkylaminocarbonyl, C₁-C₄ alkylaminosulphonyl, C₃-C₇ cycloalkyl, phenyl, 5- to 10-membered heterocyclyl and 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
and
R²² represents C₃-C₇ cycloalkyl, phenyl, 5- to 10-membered heterocyclyl or 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
and
whereby
o represents a number 0, 1, 2 or 3,
P represents a number 0, 1, 2 or 3,
q represents a number 2 or 3,
v represents a number 2 or 3,
R¹⁶ represents C₁-C₆ alkyl, C₂-C₄ alkenyl, C₁-C₆ alkoxy, phenyl, benzyloxy or 5- to 10-membered heterocyclyl, whereby alkyl, alkenyl and alkoxy can be substituted with a substituent, whereby the substituent is selected from the group consisting of halogen, cyano, hydroxy, trifluoromethyl, hydroxycarbonyl, aminosulphonyl, C₁-C₄ alkoxy, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
R¹⁷ represents hydrogen, C₁-C₄ alkyl or phenyl,
whereby alkyl can be substituted by a substituent, whereby the substituent is selected from the group consisting of methoxy, methoxycarbonyl, C₃-C₇ cycloalkyl, phenyl, 5- to 10-membered heterocyclyl and 5- to 10-membered heteroaryl,
wherein cycloalkyl, phenyl, heterocyclyl and heteroaryl for their part can be substituted with 1 to 3 substituents, whereby the substituents are selected independently of one another from the group consisting of halogen, cyano, hydroxy, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl, C₁-C₄ alkylsulphonylamino and C₁-C₄ alkoxycarbonyl,
R¹⁸ represents water or C₁-C₄ alkyl,
R²⁴ represents water or C₁-C₄ alkyl,
R²⁵ represents C₁-C₆ alkyl, C₂-C₄ alkenyl, C₁-C₆ alkoxy, phenyl, benzyloxy or 5- to 10-membered heterocyclyl, whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
R²⁶ represents hydrogen or C₁-C₄ alkyl,
R²⁷ represents C₁-C₆ alkyl, C₂-C₄ alkenyl, phenyl or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
Y represents an oxygen atom, a sulphur atom or NR¹⁵,
whereby
R¹⁵ represents C₁-C₆ alkyl, C₂-C₄ alkenyl, C₁-C₄ alkylsulphonyl, benzylsulphonyl, —(CH₂)ᵣCOR¹⁹, —(CH₂)ₛCONR²⁰R²¹, —(CH₂)ₜNR²⁸COR²⁹ or —(CH₂)_{w}NR³⁰SO₂R³¹,
whereby alkyl, alkenyl and alkylsulphonyl can be substituted with 1 to 2 substituents, whereby the substituents are selected independently of one another from the group consisting of halogen, cyano, hydroxy, trifluoromethyl, hydroxycarbonyl, aminosulphonyl, C₁-C₄ alkoxy, C₁-C₄ alkoxycarbonyl, C₁-C₆ alkylaminocarbonyl, C₁-C₆ alkylaminosulphonyl, benzylaminosulphonyl, C₃-C₇ cycloalkyl, phenyl, 5- to 10-membered heterocyclyl, 5- to 10-membered heteroaryl and —OR²³,
wherein phenyl, heterocyclyl and heteroaryl can be substituted with 1 to 3 substituents, whereby the substituents are selected independently of one another from the group consisting of halogen, cyano, nitro, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl, C₁-C₄ alkoxycarbonyl and benzyl,
and
wherein alkoxy can be substituted with a substituent selected from the group consisting of halogen, cyano, trifluoromethyl, hydroxy, hydroxycarbonyl, aminocarbonyl, aminosulphonyl, C₁-C₄ alkoxy, C₁-C₄ alkoxycarbonyl, C₁-C₄ alkylaminocarbonyl, C₁-C₄ alkylaminosulphonyl, C₃-C₇ cycloalkyl, phenyl, 5- to 10-membered heterocyclyl and 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
and
R²³ represents C₃-C₇ cycloalkyl, phenyl, 5- to 10-membered heterocyclyl or 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
and
whereby
r represents a number 0, 1, 2 or 3,
s represents a number 0, 1, 2 or 3,
t represents a number 2 or 3,
w represents a number 2 or 3,
R¹⁹ represents C₁-C₆ alkyl, C₂-C₄ alkenyl, C₁-C₆ alkoxy, phenyl, benzyloxy or 5- to 10-membered heterocyclyl, whereby alkyl, alkenyl and alkoxy can be substituted with a substituent, whereby the substituent is selected from the group consisting of halogen, cyano, hydroxy, trifluoromethyl, hydroxycarbonyl, aminosulphonyl, C₁-C₄ alkoxy, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
R²⁰ represents hydrogen, C₁-C₄ alkyl or phenyl,
whereby alkyl can be substituted with a substituent, whereby the substituent is selected from the group consisting of methoxy, methoxycarbonyl, C₃-C₇ cycloalkyl, phenyl, 5- to 10-membered heterocyclyl and 5- to 10-membered heteroaryl,
wherein cycloalkyl, phenyl, heterocyclyl and heteroaryl for their part can be substituted with 1 to 3 substituents, whereby the substituents are selected independently of one another from the group consisting of halogen, cyano, hydroxy, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl, C₁-C₄ alkylsulphonylamino and C₁-C₄ alkoxycarbonyl,
R²¹ represents hydrogen or C₁-C₄ alkyl,
R²⁸ represents hydrogen or C₁-C₄ alkyl,
R²⁹ represents C₁-C₆ alkyl, C₂-C₄ alkenyl, C₁-C₆ alkoxy, phenyl, benzyloxy or 5- to 10-membered heterocyclyl, whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
R³⁰ represents hydrogen or C₁-C₄ alkyl,
R³¹ represents C₁-C₆ alkyl, C₂-C₄ alkenyl, phenyl or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
R⁸ represents hydrogen, oxo, trifluoromethyl, trifluoromethoxy, C₁-C₄ alkyl, C₁-C₄ alkoxy or C₁-C₄ alkylthio,
R⁹ represents hydrogen, C₁-C₄ alkyl or C₁-C₄ alkoxy,
R¹⁰ represents hydrogen, C₁-C₄ alkyl,
R¹¹ represents hydrogen, C₁-C₄ alkyl,
R¹² represents hydrogen, C₁-C₄ alkyl,
R¹³ represents hydrogen, C₁-C₄ alkyl,
R³ represents hydrogen, halogen, cyano, methyl, methoxy, ethoxy or phenoxy,
R⁴ represents hydrogen, halogen, methyl, methoxy or ethoxy,
R⁵ represents hydrogen, halogen, cyano, nitro, hydroxy, amino, trifluoromethyl, trifluoromethoxy, hydroxycarbonyl, aminocarbonyl, hydroxymethyl, aminomethyl, C₁-C₄ alkyl, C₁-C₄ alkoxy, C₁-C₄ alkylamino, C₁-C₄ alkylthio, C₁-C₄ alkylcarbonyl, C₁-C₄ alkoxycarbonyl, C₁-C₄ alkylaminocarbonyl, C₃-C₆ cycloalkylaminocarbonyl, C₁-C₄ alkylcarbonylamino, C₁-C₄ alkoxycarbonylamino, C₁-C₄ alkylsulphonyl, C₁-C₄ alkylsulphonylamino, C₂-C₄ alkenylsulphonylamino, C₁-C₄ alkylsulphonyl(C₁-C₄ alkyl)amino, benzylsulphonylamino, 5- or 6-membered heteroarylsulphonylamino or 5- to 7-membered heterocyclyl,
whereby alkylaminocarbonyl, alkylcarbonylamino and alkylsulphonylamino can be substituted with a substituent, whereby the substituent is selected from the group consisting of cyano, hydroxy, amino, hydroxycarbonyl, C₁-C₄ alkoxy, C₁-C₄ alkylamino, morpholinyl, piperidinyl, pyrrolidinyl and benzylamino,
R⁶ represents hydrogen, halogen, C₁-C₄ alkyl or C₁-C₄ alkoxy,
R⁷ represents hydrogen, halogen, C₁-C₄ alkyl or C₁-C₄ alkoxy,
Or
R⁵ and R⁶ are bonded to neighbouring carbon atoms and together with the carbon atoms to which they are bonded form a dioxolane ring,
or one of their salts, their solvates or the solvates of their salts.

2. Compound according to claim 1, wherein
R¹ and R² together with the carbon atom to which they are bonded form a compound of formula 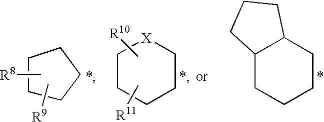
whereby
* represents the carbon atom to which R¹ and R² are bonded,
n represents the number 2,
X represents an oxygen atom, a sulphur atom or NR¹⁴,
whereby
R¹⁴ represents C₁-C₆ alkyl, C₂-C₄ alkenyl, C₁-C₄ alkylsulphonyl, benzylsulphonyl, —(CH₂)ₒCOR¹⁶ or —(CH₂)ₚCONR¹⁷R¹⁸,
whereby alkyl, alkenyl and alkylsulphonyl can be substituted with 1 to 2 substituents, whereby the substituents are selected independently of one another from the group consisting of halogen, cyano, hydroxy, trifluoromethyl, hydroxycarbonyl, aminosulphonyl, C₁-C₄ alkoxy, C₁-C₄ alkoxycarbonyl, C₃-C₇ cycloalkyl, phenyl, 5- to 10-membered heterocyclyl and 5- to 10-membered heteroaryl,
wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
and
whereby
o represents a number a number 0, 1, 2 or 3,
P represents a number a number 0, 1, 2 or 3,
R¹⁶ represents C₁-C₆ alkyl, C₂-C₄ alkenyl, C₁-C₆ alkoxy, phenyl, benzyloxy or 5- to 10-membered heterocyclyl, whereby alkyl and alkenyl can be substituted with a substituent, whereby the substituent is selected from the group consisting of halogen, cyano, hydroxy, trifluoromethyl, hydroxycarbonyl, aminosulphonyl, C₁-C₄ alkoxycarbonyl, C₃-C₇ cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
R¹⁷ represents hydrogen, C₁-C₄ alkyl or phenyl,
wherein alkyl can be substituted with a substituent, whereby the substituent is selected from the group consisting of methoxy, C₃-C₇ cycloalkyl, phenyl, 5- to 10-membered heterocyclyl and 5- or 6-membered heteroaryl,
wherein phenyl and heteroaryl for their part can be substituted with 1 to 3 substituents, whereby the substituents are selected independently of one another from the group consisting of halogen, cyano, trifluoromethyl, trifluoromethoxy and C₁-C₄ alkyl,
R¹⁸ represents hydrogen or C₁-C₄ alkyl,
R⁸ represents hydrogen, oxo, trifluoromethyl, trifluoromethoxy, C₁-C₄ alkyl, C₁-C₄ alkoxy or C₁-C₄ alkylthio,
R⁹ represents hydrogen, C₁-C₄ alkyl or C₁-C₄ alkoxy,
R¹⁰ represents hydrogen,
R¹¹ represents hydrogen,
R³ represents hydrogen, halogen, methyl, methoxy, ethoxy or phenoxy,
R⁴ represents hydrogen, halogen, methyl, methoxy, ethoxy,
R⁵ represents hydrogen, halogen, cyano, nitro, hydroxy, amino, trifluoromethyl, trifluoromethoxy, hydroxycarbonyl, aminocarbonyl, hydroxymethyl, aminomethyl, C₁-C₄ alkyl, C₁-C₄ alkoxy, C₁-C₄ alkylamino, C₁-C₄ alkylthio, C₁-C₄ alkylcarbonyl, C₁-C₄ alkylaminocarbonyl, C₃-C₆ cycloalkylaminocarbonyl, C₁-C₄ alkylcarbonylamino, C₁-C₄ alkoxycarbonylamino, C₁-C₄alkylsulphonyl, C₁-C₄ alkylsulphonylamino, C₂-C₄ alkenylsulphonylamino, C₁-C₄ alkylsulphonyl(C₁-C₄ alkyl)amino, benzylsulphonylamino, 5- or 6-membered heteroarylsulphonylamino or 5- to 7-membered heterocyclyl,
whereby alkylaminocarbonyl, alkylcarbonylamino and alkylsulphonylamino can be substitutes with a substituent, whereby the substituent is selected from the group consisting of cyano, hydroxy, amino, hydroxycarbonyl, C₁-C₄ alkoxy, C₁-C₄ alkylamino, morpholinyl, piperidinyl, pyrrolidinyl and benzylamino,
R⁶ represents hydrogen, halogen, C₁-C₄ alkyl or C₁-C₄ alkoxy,
R⁷ represents hydrogen,
Or
R⁵ and R⁶ are bonded to neighbouring carbon atoms and together with the carbon atom to which they are bonded form a 1,3-dioxolane,
or one of their salts, solvates or solvates of their salts.

3. Compound according to claim 1, wherein
R¹ and R² together with the carbon atom to which they are bonded form a group of the formula 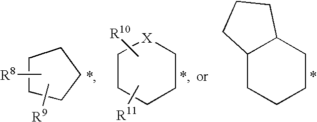
whereby
* represents the carbon atom to which R¹ and R² are bonded,
n represents the number 2,
X represents NR¹⁴,
whereby
R¹⁴ represents C₁-C₄ alkyl, C₂-C₄ alkenyl, benzylsulphonyl, —(CH₂)ₒCOR¹⁶ or —(CH₂)ₚCONR¹⁷R¹⁸,
whereby alkyl and alkenyl can be substituted with 1 to 2 substituents, whereby the substituents are selected independently of one another from the group consisting of halogen, cyano, hydroxy, trifluoromethyl, hydroxycarbonyl, aminosulphonyl, C₁-C₄ alkoxy, C₁-C₄ alkoxycarbonyl, C₃-C₇ cycloalkyl, phenyl, 5- to 10-membered heterocyclyl and 5- to 10-membered heteroaryl,
wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulphonyl, C₁-C₄ alkyl, C₁-C₄ alkoxy, C₁-C₄ alkylamino, C₁-C₄ alkylsulphonyl and C₁-C₄ alkoxycarbonyl,
and
whereby
o represents a number 1 or 2,
p represents a number 1 or 2,
R¹⁶ represents C₁-C₄ alkyl, C₁-C₄ alkoxy, phenyl or benzyloxy,
R¹⁷ for hydrogen, C₁-C₄ alkyl or phenyl,
whereby alkyl can be substituted with a substituent, whereby the substituent is selected from the group consisting of methoxy, phenyl and 5- or 6-membered heteroaryl,
wherein phenyl for its part can be substituted with 1 to 3 substituents, whereby the substituents are selected independently of one another from the group consisting of halogen, cyano, trifluoromethyl, trifluoromethoxy and C₁-C₄ alkyl,
R¹⁸ represents hydrogen,
R⁸ represents hydrogen, C₁-C₄ alkyl or C₁-C₄ alkoxy,
R⁹ represents hydrogen or C₁-C₄ alkyl,
R¹⁰ represents hydrogen,
R¹¹ represents hydrogen,
R³ represents hydrogen, halogen, methyl, ethoxy or phenoxy,
R⁴ represents hydrogen, halogen or methyl,
R⁵ represents hydrogen, halogen, cyano, hydroxy, trifluoromethyl, trifluoromethoxy, hydroxycarbonyl, aminocarbonyl, hydroxymethyl, C₁-C₄ alkyl, C₁-C₄ alkoxy, C₁-C₄ alkylaminocarbonyl, C₃-C₆ cycloalkylaminocarbonyl, C₁-C₄ alkylcarbonylamino, C₁-C₄ alkoxycarbonylamino, C₁-C₄ alkylsulphonyl, C₁-C₄ alkylsulphonylamino, C₂-C₄ alkenylsulphonylamino, C₁-C₄ alkylsulphonyl(C₁-C₄ alkyl)amino, benzylsulphonylamino or 5- or 6-membered heteroarylsulphonylamino,
whereby alkylaminocarbonyl, alkylcarbonylamino and alkylsulphonylamino can be substituted with a substituent, whereby the substituent is selected from the group consisting of amino, C₁-C₄ alkylamino, morpholinyl and pyrrolidinyl,
R⁶ represents hydrogen, halogen, C₁-C₄ alkyl or C₁-C₄ alkoxy,
R⁷ represents hydrogen,
or one of their salts, their solvates or solvates of their salts.

4. A method for the preparation of a compound of formula according to claim 1, wherein according to method
[A] a compound of formula 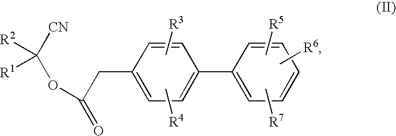
in which
R¹, R², R³, R⁴, R⁵, R⁶ and R⁷ have the meaning indicated in claim 1
is reacted with a base,
Or
[B] a compound of formula 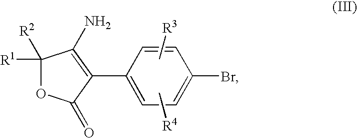
in which
R¹, R², R³ and R⁴ have the meaning indicated in claim 1
is reacted under Suzuki coupling conditions with a compound of the formula 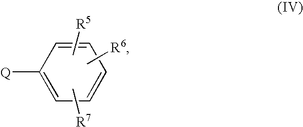
in which
R⁵, R⁶ and R⁷ have the meaning indicated in claim 1, and
Q represents —B(OH)₂, a boronic acid ester —BF₃³¹ K⁺.

5. A compound according to claim 1, for the treatment and/or prophylaxis of diseases.

6. A pharmaceutical composition comprising at least one compound according to claim 1 in combination with at least one inert, non-toxic, pharmaceutically acceptable excipient.

7. (canceled)

8. The pharmaceutical composition according to claim 6 for the treatment and/or prophylaxis of viral diseases.

9. The pharmaceutical composition according to claim 8 for the treatment and/or prophylaxis of HIV infections.

10. A method for controlling viral diseases in humans and animals by the administration of an anti-virally effective amount of at least one compound according to claim 1.

11. A method for controlling viral diseases in humans and animals by the administration of an anti-virally effective amount of a pharmaceutical composition of claim 6.
